(19) United States
(12) Patent Application Publication
(10) Pub. No.: US 2013/0052259 A1
(43) Pub. Date: Feb. 28, 2013

(54) LIPOSOMES COMPRISING AMPHIPATHIC DRUGS AND METHOD FOR THEIR PREPARATION

(71) Applicants: Yechezkel Barenholz, Jerusalem (IL); Daniel Zucker, Kyriat Bialik (IL)

(72) Inventors: Yechezkel Barenholz, Jerusalem (IL); Daniel Zucker, Kyriat Bialik (IL)

(21) Appl. No.: 13/576,570

(22) PCT Filed: Feb. 1, 2011
(86) PCT No.: PCT/IL2011/000114

§ 371 Date: Oct. 22, 2012

Related U.S. Application Data
(60) Provisional application No. 61/300,181, filed on Feb. 1, 2010.

(51) Int. Cl. A61K 9/127; A61P 35/00; A61K 31/4745

(52) U.S. Cl. 424/450; 514/283; 264/4.1

(57) ABSTRACT

The present invention provides a liposome having co-encapsulated in its intraliposomal aqueous core at least two amphipathic drugs, the liposomes being characterized by one of the following: the amphipathic drugs are co-encapsulated at a pre-determined ratio; the liposome comprises one or a combination of liposome forming lipids have a solid ordered to liquid disordered phase transition temperature above 37° C.; each of the amphipathic drugs exhibit a liposomal profile that corresponds to the profile of each drug when encapsulated as a single drug in the same liposome; and the liposome is absent of one or both of a transition metal and a ionophore. The invention also provides a method for preparing such liposomes. This method, taken together with the features of the liposomal composition, provides high loading and long term stability of the resulting co-encapsulated liposomal formulation.

### FIELD OF THE INVENTION

This invention relates to liposomes technology and in particular to liposomes having encapsulated thereon at least two drugs.

### BACKGROUND OF THE INVENTION

The use of liposomes and nanoliposomes may improve the therapeutic index of drugs by: (1) selective delivery serving as a device for controlled release of drugs, (2) reducing exposure of sensitive tissue to toxic drugs, and (3) controlling the drug's pharmacokinetics and biodistribution. The nano range (diameter≦100 nm) due to the enhanced permeability and retention (EPR) effect causes tumor-selective localization of the nanoliposomes. Drug release at the tumor site is related to the effect of the unique tumor environment on the liposome membrane and/or the gradient that stabilizes the loading.

It was demonstrated that in vivo maintenance of drug ratios shown to be synergistic in vitro provided increased efficacy in preclinical tumor models, whereas attenuated antitumor activity was reported when antagonistic drug ratios were maintained for the combinations of irinotecan/floxuridine, cytarabine/daunorubicin, and cisplatin/daunorubicin (G. Batist, K. A. Gelmon, K. N. Chi, W. H. Miller, Jr., S. K. Chia, L. D. Mayer, C. E. Swenson, A. S. Janoff, A. C. Louie, Safety, pharmacokinetics, and efficacy of CPX-1 liposome injection in patients with advanced solid tumors. Clin Cancer Res 15(2) (2009) 692-700; L. D. Mayer, T. O. Harasym, P. G. Tardi, N. L. Harasym, C. R. Shew, S. A. Johnstone, E. C. Ramsay, M. B. Bally, A. S. Janoff, Ratiometric dosing of anticancer drug combinations: controlling drug ratios after systemic administration regulates therapeutic activity in tumor-bearing mice. Mol Cancer Ther 5(7) (2006) 1854-1863; P. Tardi, R. Gallagher, S. Johnstone, N. Harasym, M. Webb, M. Bally, L. Mayer, Coencapsulation of irinotecan and floxuridine into low cholesterol-containing liposomes that coordinate drug release in vivo. Biochem. Biophys. Acta 1768 (2007) 678-687; P. Tardi, S. Johnstone, N. Harasym, S. Xie, T. Harasym, N. Zisman, P. Harvie, D. Bermudes, L. Mayer, In vivo maintenance of synergistic cytarabine: daunorubicin ratios greatly enhances therapeutic efficacy. Leukemia Research 33 (2009) 129-139).

Co-encapsulation of two amphipathic drugs was described where the drugs are encapsulated into the liposomes in two stages [X. Li, W. L. Lu, G. W. Liang, G. R. Ruan, H. Y. Hong, C. Long, Y. T. Zhang, Y. Liu, J. C. Wang, X. Zhang and Q. Zhang Effect of stealthy liposomal topotecan plus amlodipine on the multidrug-resistant leukaemia cells in vitro and xenograft in mice European Journal of Clinical Investigation (2006) 36, 409-418].

Further, co-encapsulated of two amphipathic drugs by remote loading was also described [JianCheng WANG, BoonCher GOH, WanLiang LU, Qiang ZHANG, Alex CHANG, Xiao Yan LIU, Theresa M C TAN, and HowSung LEE, In Vitro Cytotoxicity of Stealth Liposomes Co-encapsulating Doxorubicin and Verapamil on Doxorubicin-Resistant Tumor Cells Biol. Pharm. Bull. (2005) 28(5) 822-828].

### SUMMARY OF THE INVENTION

The present invention is based on the finding that by remote loading of two amphipathic drugs into the same nano sterically stabilized liposome (nSSL) at high loading (above 85% and preferably above 90% and at times even above 95%) of both drugs, and at a predefined drug ratio, where each drug exhibit a behavior in the liposome as if it was encapsulated alone. The two drugs also exhibit a release profile whereby the predefined ratio is essentially retained at the target site, for at least a period of time significant to achieve simultaneous and therapeutically significant effect of the drugs at the target site. In other words, the drugs reach the target site, e.g. the tumor, simultaneously at the predefined ratio, and exhibit for each drug a release profile similar to that of the drug when encapsulated alone (in separate liposomes).

The combination of two drugs in the same liposome is of particular interest in the field of cancer treatment since many curative cancer treatment regimens utilize drug combinations. The combination of two drugs in the same liposomes allows the simultaneous effect of the two drugs on different cells at the target site. Interestingly, only little work was undertaken to deliver drug combinations in liposomes. This may stem from difficulties with providing efficient and stable encapsulation of two chemotherapeutics inside a single liposome.

Thus, in accordance with a first aspect, the present invention provides a liposome having co-encapsulated in its intraliposomal aqueous core at least two amphipathic drugs, the at least two amphipathic drugs being either at least two amphipathic weak base drugs or at least two amphipathic weak acid drugs, the at least two amphipthic drugs being within the intraliposomal core, wherein
the at least two amphipathic drugs are co-encapsulated in the liposome at a pre-determined ratio;
the liposome comprises one or combination of liposome forming lipids, the one or combination of liposome forming lipids have a solid ordered (SO) to liquid disordered (LD) phase transition temperature above 37° C.;
each of the at least two amphipathic drugs exhibit a liposomal profile that corresponds to the profile of each drug when encapsulated as a single drug in the same liposome; and
the liposome is absent of one or more of a transition metal and a ionophore (i.e. one or both being absent).

In accordance with a second aspect, there is provided a method for simultaneous co-enacpsulation into a liposome of at least two amphipathic drugs, the method comprising: (a) providing a suspension of liposomes comprising in the intraliposomal aqueous core of the liposome a weak acid or weak base and a counter ion of the weak acid or weak base, the concentration of the weak acid or weak base being greater inside the liposome than outside the liposome; (b) simultaneously incubating the liposomes with at least two amphipathic drugs having a pre-determined ratio therebetween, the at least two amphipathic drugs being compatible with the counter ion, when the liposomes comprise a weak acid, the at least two amphipathic drugs are weak amphipathic acid drugs, and when the liposomes comprise a weak base, the at least two amphipathic drugs are weak amphipathic base drugs; wherein,
the liposome comprises one or combination of liposome forming lipids, the one or combination of liposome forming lipids have a solid ordered (SO) to liquid disordered (LD) phase transition temperature above 37° C.;
the incubation is under conditions sufficient to allow simultaneous co-encapsulation in the intraliposomal aqueous core of the liposome of the two amphipathic drugs without use of a transition metal and the encapsulation is at a pre-determined ratio between the at least two amphipathic drugs;
when in the liposome, each of the at least two amphipathic drugs exhibit a liposomal profile that corresponds to the profile of each drug when encapsulated as a single drug in the same liposome; and
for each drug, the method provides a loading efficiency above 85%.

In accordance with an additional aspect, there is provided a package comprising a liposomes according to the invention or a pharmaceutical composition comprising the same, and instructions for administration of the liposome or pharmaceutical composition to a subject in need thereof.

Yet, provided by the invention is the use of liposomes according to the invention, for the preparation of a pharmaceutical composition for the treatment of a condition for which at least one of the weak amphipathic drug is known to be effective.

### BRIEF DESCRIPTION OF THE DRAWINGS

In order to understand the invention and to see how it may be carried out in practice, embodiments will now be described, by way of non-limiting example only, with reference to the accompanying drawings, in which:

FIGS. 1A-1D are graphs showing the in vitro activity of vincristine (VCR), topotecan (TPT) and TPT/VCR ratios in Daoy, NB-EB and SW480 cells; where combination of fixed TPT/VCR mole ratios were titrated to provide a broad range of cell growth inhibition, reflected by fₐ, and VCR and TPT concentrations varied in the range of 1-480 and 14-650 nm respectively; points are average values from triplicate assays repeated a minimum of thrice, where Combination Index (CI) values of <1, ˜1 and >1 indicate synergy, additivity and antagonism, respectively. FIG. 1A shows IC₅₀ values (nM) of VCR and TPT in Daoy, NB-EB and SW480 cells; FIGS. 1B-1D show CI values, where FIG. 1B shows TPT/VCR ratios 73 (Δ), 14.6 (⋄), and 2.9 (▪) tested in Daoy cells; FIG. 1C shows TPT/VCR ratios tested in NB-EB neuroblastoma cells, 11.8 (Δ), 2.4 (⋄), 0.5 (▪), and 47 (); FIG. 1D shows TPT/VCR ratios tested in SW480 colon adenocarcinoma cells, 18 (Δ), 3.7 (⋄), 0.7 (▪), and 0.2 ().

FIGS. 2A-2D are graphs showing the characterization of TPT and VCR remote loading into nanoliposomes at 55° C. for 30 min under various experimental conditions: FIG. 2A shows the dependency of the loading efficiency on external medium (saline) pH, initial mole ratios were TPT/PL=0.2 and VCR/PL=0.1 and counter ion was sulfate; FIG. 2B shows the dependency of the loading efficiency on the ammonium counter ion, initial mole ratios were TPT/PL=0.2, VCR/PL=0.1, the external medium was saline at pH 6. FIG. 2C: The dependency of the % drug encapsulation and final drug/PL ration by varying the initial drug/PL ratios. The external medium was saline at pH 6 and the counter ion was sulfate. % TPT loading (▪), TPT final drug/PL ratio (▾), VCR loading % (▴), VCR final drug/PL ratio (♦); FIG. 2D The dependency of the loading efficiency on the type of the external medium; the initial mole ratios were TPT/PL=0.2, VCR/PL=0.15, the external medium pH was 6 and counter ion was sulfate.

FIGS. 3A-3D are Cryo-TEM micrographs of various liposomal formulations. FIG. 3A: micrograph of liposomes in the absence of drug, FIG. 3B micrograph of liposomal VCR at drug/PL ratio of 0.49, (C) FIG. 3C micrograph of liposomal TPT at drug/PL 0.2, FIG. 3D micrograph of LipoViTo at mole drug/PL of 0.21 and 0.28 for VCR and TPT, respectively. The size bar represents 100 nm.

FIG. 4 is a graph showing the kinetics of in vitro release of encapsulated TPT (dark lines) and encapsulated VCR (gray lines) from liposomes encapsulated with one or two drugs in adult bovine serum diluted 1:10.

FIGS. 5A-5D are graphs showing TPT and VCR concentrations and drug ratios in the plasma (FIGS. 5A-6B) or in Daoy tumors (FIG. 5C-5D) of nude mice after i.v. administration of free drugs or drugs encapsulated in liposomes, where FIG. 5A and FIG. 5C show the concentrations in the plasma and in the tumors, respectively of TPT and VCR following administration of free TPT (10 mg/kg, □), free VCR (2 mg/kg, ◯), liposomal TPT (5 mg/kg ▾) and liposomal VCR (2 mg/kg ♦); while FIG. 6B and FIG. 5D show TPT/VCR mole ratios in the plasma and in the tumors, respectively, following simultaneous i.v. administration of both drugs, with an initial administration mole ratio of TPT/VCR of 2.9 as: free drugs (), LipoViTo (e) and a mixture of liposomal TPT with liposomal VCR (▾).

FIG. 6A-6D are Kaplan Meir graphs showing the efficacy of free TPT and VCR or delivered in nSSL against solid tumors models. The doses of the single agent treatments were identical in all experiments; free VCR-2 mg/kg, nSSL VCR-2 mg/kg, free TPT-10 mg/kg, nSSL TPT-5 mg/kg; FIG. 6A shows Medulloblastoma treated by free VCR, nSSL VCR, free TPT, nSSL TPT, free synergistic drugs-TPT 2.7 mg/kg and VCR 2 mg/kg, synergistic LipoViTo-TPT 2.7 mg/kg and VCR 2 mg/kg, antagonistic LipoViTo-TPT 5 mg/kg and VCR 0.15 mg/kg, two liposomes given together-nSSL TPT 2.7 mg/kg and nSSL VCR 2 mg/kg (synergistic ratio); FIG. 6B shows. colon cancer treated by free VCR, nSSL VCR, free TPT, nSSL TPT, free synergistic drugs-TPT 5 mg/kg and VCR 0.552 mg/kg, synergistic LipoViTo-TPT 5 mg/kg and VCR 0.552 mg/kg, antagonistic LipoViTo-TPT 0.736 mg/kg and VCR 2 mg/kg, two liposomes given together-nSSL TPT 5 mg/kg and nSSL VCR 0.552 mg/kg (synergistic ratio); FIG. 6C shows Medulloblastoma treated by synergistic LipoViTo-TPT 2.7 mg/kg and VCR 2 mg/kg and MTD LipoViTo-TPT 5 mg/kg and VCR 1.5 mg/kg; and FIG. 6D shows colon cancer treated by synergistic LipoViTo-TPT 5 mg/kg and VCR 0.552 mg/kg and MTD LipoViTo-TPT 5 mg/kg and VCR 1.5 mg/kg. Nine mice were treated in each group. All mice received injections through the tail vain at days 17, 23 and 29.

### DETAILED DESCRIPTION OF EMBODIMENTS

The present disclosure is based on a research investigating controlled drug pharmacokinetics in vivo when co-encapsulating two amphipathic drugs in the same liposome. Further investigated was the loading efficiency and control of optimal drug/drug ratios in vivo, in the aim of providing an increase in therapeutic efficacy/therapeutic index of the combined drugs, when co-encapsulated, as compared to the effect obtained when administering the two drugs in two distinct liposomes, albeit with the same liposome membrane composition.

For this purpose, the inventors have developed a methodology allowing encapsulation of two or more amphipathic drugs in the same liposome with very high loading efficacy and low leakage of the drugs from the liposomes. This was achieved using the remote loading, with the same counter-ion acting as the driving force for the two or more amphipathic drugs, for encapsulation into a liposome having a rigid membrane.

The term “high loading” denotes loading of the drug at a concentration in the intraliposomal aqueous core that is characterized by one of the following (i) a concentration in the intraliposomal aqueous core above the maximal solubility of the drug in water; (ii) a concentration in the intraliposomal aqueous core above 1.2 times the maximal solubility of the drug in water; (iii) a concentration in the intraliposomal aqueous core in the range of between 1.2 to 2.5 times the maximal solubility of the drug in water; or (iv) a concentration in the intraliposomal aqueous core above 50 mM.

Thus, in accordance with one aspect, there is provided a liposome having co-encapsulated in its intraliposomal core, at least two amphipathic drugs, the at least two amphipathic drugs being either at least two amphipathic weak base drugs or at least two amphipathic weak acid drugs, the at least two amphipthic drugs being within the intraliposomal core,

wherein,
the at least two amphipathic drugs are co-encapsulated in the liposome at a pre-determined ratio;
the liposome comprises one or combination of liposome forming lipids, the one or combination of liposome forming lipids have a solid ordered (SO) to liquid disordered (LD) phase transition temperature above 37° C. or even above 40° C.; each of the at least two amphipathic drugs exhibit a liposomal profile that corresponds to the profile of each drug when encapsulated as a single drug in the same liposome; and
the liposome being absent of a transition metal and/or a ionophore.

The term “liposome” is used herein to denote lipid based bilayer vesicles. The liposomes are those composed primarily of vesicle-forming lipids which are amphiphilic molecules essentially characterized by a packing parameter 0.74-1.0, or by a lipid mixture having an additive packing parameter (the sum of the packing parameters of each component of the liposome times the mole fraction of each component) in the range between 0.74 and 1.

“Vesicle-forming lipids”, also referred to as “liposome forming lipids” denote primarily glycerophospholipids and sphingomyelins that form into bilayer vesicles in water. The glycerophospholipids have a glycerol backbone wherein at least one, preferably two, of the hydroxyl groups at the head group is substituted by one or two of an acyl, alkyl or alkenyl chain, a phosphate group, or combination of any of the above, and/or derivatives of same and may contain a chemically reactive group (such as an amine, acid, ester, aldehyde or alcohol) at the head group, thereby providing the lipid with a polar head group. The sphingomyelins consists of a ceramide unit with a phosphorylcholine moiety attached to position 1 and thus in fact is an N-acyl sphingosine. The phosphocholine moiety in sphingomyelin contributes the polar head group of the sphingomyelin.

In the liposome forming lipids the hydrocarbon chain(s) are typically between 14 to about 24 carbon atoms in length, and have varying degrees of saturation being fully, partially or non-hydrogenated naturally occurring lipids, semi-synthetic or fully synthetic lipids and the level of saturation may affect rigidity of the liposome thus formed (typically lipids with saturated chains are more rigid than lipids of same chain length in which there are un-saturated chains (especially having cis double bonds)). Further, the lipid matrix may be of natural source or natural lipids which have been modified, semi-synthetic or fully synthetic lipid, and neutral, negatively or positively charged.

There are a variety of synthetic, semi-synthetic and naturally-occurring vesicle (liposome)-forming lipids, which may be categorized according to their charge and saturation of the hydrocarbon chain. In the context of the invention, any such vesicle-forming lipids may be utilized, as long as they fulfill the condition of forming a rigid membrane. In order to form a rigid membrane the liposome forming lipids are selected based on their solid ordered (SO) to liquid disordered (LD) phase transition temperature being Tₘ>37° C. Tₘ is the temperature within the range of the SO to LD phase transition temperatures in which the maximal change in the heat capacity of the phase transition occurs.

In line with the above condition regarding the Tₘ of the liposome forming lipids, the following one or more lipids may be used (the following Tₘ being obtained from Avanti On Line site http://www.avantilipids.com).

Neutral (zwitterionic, namely, having no net charge) lipids may be a phosphatidylcholine (PC) and derivatives thereof, such as 1,2-dipalmitoyl-sn-glycero-3-phosphocholine (DPPC, 16:0PC, Tₘ˜41.4° C.), 1,2-diheptadecanoyl-sn-glycero-3-phosphocholine (17:0PC, Tₘ˜41° C.), 1,2-distearoyl-sn-glycero-3-phosphocholine (DSPC, 18:0PC, Tₘ˜55° C.), 1,2-dinonadecanoyl-sn-glycero-3-phosphocholine (19:0PC, Tₘ˜60° C.), 1,2-diarachidoyl-sn-glycero-3-phosphocholine (DBPC, 20:0PC Tₘ˜66° C.), 1,2-dihenarachidoyl-sn-glycero-3-phosphocholine (21:0PC Tₘ˜72° C.), 1,2-dibehenoyl-sn-glycero-3-phosphocholine (22:0PC Tₘ˜75° C.), 1,2-ditricosanoyl-sn-glycero-3-phosphocholine (23:0PC Tₘ˜79° C.), 1,2-dilignoceroyl-sn-glycero-3-phosphocholine (24:0PC Tₘ˜80° C.), 1-myristoyl-2-stearoyl-sn-glycero-3-phosphocholine (14:0-18:0PC Tₘ˜40° C.), 1-palmitoyl-2-stearoyl-sn-glycero-3-phosphocholine (PSPC, 16:0-18:0PC Tₘ˜49° C.), 1-stearoyl-2-palmitoyl-sn-glycero-3-phosphocholine (SPPC, 18:0-16:0PC Tₘ˜44° C.), hydrogenated soy phosphatidylcholine (HSPC, Tₘ˜52.5° C.).

Negatively charged lipids (i.e. having a net negative charge) may include, without being limited thereto phosphatidylserine (PS) and derivatives thereof such as 1,2-dipalmitoyl-sn-glycero-3-phospho-L-serine (DPPS, 16:0 PS, Tₘ˜54° C.), brain phosphatidylserine (BPS), 1,2-distearoyl-sn-glycero-3-phospho-L-serine (DSPS, 18:0PS Tₘ˜68° C.), phosphatidylglycerol (PG) and derivatives thereof such as dilauryloylphosphatidylglycerol (DLPG), 1,2-dipalmitoyl-sn-glycero-3-phospho-(1′-rac-glycerol) (DPPG, 16:0PG, Tₘ˜41° C.), 1,2-distearoyl sn-glycero-3-phospho-(1′-rac-glycerol) (DSPG, 18:0 PG, Tₘ˜55° C.), or phosphatide acid (PA) and derivatives thereof, 1,2-dimyristoyl-sn-glycero-3-phosphate (DMPA, 14:0 PA, Tₘ˜50° C.), 1,2-dipalmitoyl-sn-glycero-3-phosphate (DPPA, 16:0PA, Tₘ˜67° C.) and 1,2-distearoyl-sn-glycero-3-phosphate (DSPA, 18:0 PA, Tₘ˜75° C.).

Cationic lipids (mono and polycationic) have an overall net positive charge. Monocationic lipids may include, for example, 1,2-dimyristoyl-3-trimethylammonium propane (DMTAP) 3β[N—(N′,N′-dimethylaminoethane)carbamoly]cholesterol (DC-Chol); and dimethyl-dioctadecylammonium (DDAB).

Polycationic lipids may include a lipophilic moiety as with the mono cationic lipids, to which polycationic moiety is attached. Exemplary polycationic moieties include ceramide carbamoyl spermine (N-palmitoyl D-erythro-sphingosyl carbamoyl-spermine, CCS).

The above-described lipids with varying degrees of saturation of the acyl chains, as desired, can be obtained commercially, e.g. from Avanti Polar Lipids Inc., or prepared according to published methods.

Other lipids suitable for liposome formation may include glycolipids and sterols, such as cholesterol. Such other lipids will not include egg PC (EPC).

The vesicle-forming lipids and their combination may be selected to achieve a specified degree of rigidity, to control the stability of the liposome in serum and to control the rate of release of the entrapped agent in the liposome. As indicated above, it is required that the liposome forming lipids provide rigidity to the resulting membrane, so as to prevent undesired leakage of the drugs from the liposomes. On the other hand, the addition of cholesterol may assist in manipulating the rigidity/fluidity as desired.

In one embodiment, the liposomes include a vesicle-forming lipid derivatized with a hydrophilic polymer known by the term lipopolymers. Lipopolymers preferably comprise lipids (preferably liposome forming lipid) modified at their head group with a polymer having a molecular weight equal or above 750 Da. The head group may be polar or apolar, however, is preferably a polar head group to which a large (>750 Da) highly hydrated (at least 60 molecules of water per head group) flexible polymer is attached. The attachment of the hydrophilic polymer head group to the lipid region may be a covalent or non-covalent attachment, however, is preferably via the formation of a covalent bond (optionally via a linker).

The outermost surface coating of hydrophilic polymer chains is effective to provide a liposome with a long blood circulation lifetime in vivo. The inner coating of hydrophilic polymer chains extends into the aqueous compartments in the liposomes, i.e., between the lipid bilayers and into the central core compartment, and is in contact with any entrapped agents.

Preparation of vesicles composed of liposome-forming lipids and derivatization of such lipids with hydrophilic polymers (thereby forming lipopolymers) has been described, for example by Tirosh et al. [Tirosh et al., Biopys. J., 74(3):1371-1379, (1998)] and in U.S. Pat. Nos. 5,013,556; 5,395,619; 5,817,856; 6,043,094, 6,165,501, incorporated herein by reference and in WO 98/07409. The lipopolymers may be non-ionic lipopolymers (also referred to at times as neutral lipopolymers or uncharged lipopolymers) or lipopolymers having a net negative or a net positive charge.

There are numerous polymers which may be attached to lipids. Polymers typically used as lipid modifiers include, without being limited thereto: polyethylene glycol (PEG), polysialic acid, polylactic (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. The polymers may be employed as homopolymers or as block or random copolymers.

While the lipids derivatized into lipopolymers may be neutral, negatively charged, as well positively charged, i.e. there is not restriction to a specific (or no) charge. For example the neutral distearoyl glycerol and the negatively charged distearoyl phosphatidylethanolamine, both covalently attached to methoxy poly(ethylene glycol) (mPEG or PEG) of Mw 750, 2000, 5000, or 12000 [Priev A, et al. Langmuir 18, 612-617 (2002); Garbuzenko O., Langmuir 21, 2560-2568 (2005)]. The most commonly used and commercially available lipids derivatized into lipopolymers are those based on phosphatidyl ethanolamine (PE), usually, distearylphosphatidylethanolamine (DSPE).

A specific family of lipopolymers employed by the invention include methoxy PEG-DSPE (with different lengths of PEG chains) in which the PEG polymer is linked to the DSPE primary amino group via a carbamate linkage. The PEG moiety preferably has a molecular weight of the head group is from about 750 Da to about 20,000 Da. More preferably, the molecular weight is from about 750 Da to about 12,000 Da and most preferably between about 1,000 Da to about 5,000 Da. One specific PEG-DSPE employed herein is that wherein PEG has a molecular weight of 2000 Da, designated herein ²⁰⁰⁰PEG-DSPE or ^{2k}PEG-DSPE.

In one embodiment the liposomes's bilayer comprise at least one phospholipid, a lipopolymer and a sterol. According to a specific example in this embodiment the liposomes comprise in their bilayer, at least PC or PC derivative, PEG-derivatized lipid, and cholesterol.

A preferred embodiment comprises a liposome comprising at least PC selected from the group consisting of hydrogenated soybean phosphatidylcholime (HSPC), Dipalmitoylphosphatidylcholine (DPPC), a lipopolymer of 1,2-distearoyl-sn-glycero-3-phosphoethanolamine-N-[methoxy(polyethylene glycol)-2000] (^{2k}PEG-DSPE) and cholesterol.

An alternative to PEGylated lipids are phosphatidyl polyglycerols, which may also be used as a lipopolymer in accordance with the present disclosure. A particular example may include dipalmitoylphosphatidylpolyglycerol (DPP-PG) of different chain lengths.

In some embodiments, the mole ratio between the liposome components phosphoplipid/cholesterol/lipopoylmer is between 65:25:10 and 45:50:5.

In some embodiments, the liposomes may be formed without a lipopolymer, for example, small liposomes formed from sphingomyelin and cholesterol. Further, liposomes may be formed without a lipopolymer, for example, small liposomes formed from saturated phosphatidyl glycerol.

A preferred embodiment of the invention refers to liposomes comprising a combination of hydrogenated soybean phosphatidylcholime (HSPC), 1,2-distearoyl-sn-glycero-3-phosphoethanolamine-N-[methoxy(polyethylene glycol)-2000] (^{2k}PEG-DSPE) and cholesterol. The ratio between these components may vary within the defined range. However, according to one embodiment, the mole ratio between the components HSPC/Chol/^{2k}PEG-DSPE is about 54:41:5.

Liposomes are categorized according to the number of lamellae and size. Small vesicles show a diameter of 20 to approximately 100 nm. Large vesicles, multilamellar vesicles, and multivesicular vesicles range in size from a few hundred nanometers to several microns. The thickness of the membrane (phospholipid bilayer) measures approximately 5 to 6 nm.

In accordance with an embodiment of the present disclosure, the liposomes have a size of between 20 nm and 150 nm, even between 50 nm and 120 nm and even between 70 nm to 100 nm. This embodiment is of particular interest for systemic delivery of the drugs.

Small vesicles can be created by sonication which is process involving disruption of large multilamellar vesicle suspensions using sonic energy (sonication). The most common instrumentation for preparation of sonicated particles are bath and probe tip sonicators. Cup-horn sonicators, although less widely used, have successfully produced small vesicles. In this technique, the liposome contents are the same as the contents of the aqueous phase. Small vesicles may also be formed by extrusion of multilamellar vesicles which are forced through small orifices, such as a polycarbonate filter with a defined pore size to yield particles having a diameter near the pore size of the filter used. Prior to extrusion through the final pore size, multilamellar vesicles suspensions may be disrupted either by several freeze-thaw cycles or by pre-filtering the suspension through a larger pore size (typically 0.2 μm-1.0 μm).

Liposomes in the size range of between 100 nm and 200 nm can be prepared by a variety of methods including extrusion, detergent removal technique/dialysis (Di-Octylglucoside Vesicles or DOV), fusion of small vesicles (Fused, Unilamellar Vesicles or FUV), reverse evaporation (Reverse Evaporation Vesicles or REV), Calcium-Induced Fusion Method, ethanol or ether injection; extrusion under nitrogen through polycarboriatefilters.

In yet another embodiment, the liposomes are in the size range of 500 nm to 30 μm, e.g. for local delivery of the liposomes and the drugs encapsulated therein.

Liposomes are characterized by an intraliposomal aqueous phase (core) where a therapeutic agent may be encapsulated.

The term “encapsulating” is used herein to denote the entrapment of the at least two amphipathic drugs in the aqueous phase of the vesicle, e.g. in the intraliposomal aqueous core of the liposome.

The term “amphipathic” is used herein to denote a compound containing both polar and nonpolar domains and thus having the ability to permeate normally nonpermeable membrane under suitable conditions.

The term “amphipathic weak acid” is used herein to denote a molecule having both hydrophobic (nonpolar) and hydrophilic (polar) groups, and being characterized by any one of the following:
pKa: it has a pKa above 3.0, preferably above 3.5, more preferably, in the range between about 3.5 and about 6.5;
Partition coefficient: in an n-octanol/buffer (aqueous phase) system having a pH of 7.0, it has a logD in the range between about −3 and about 2.5.

The term “amphipathic weak base” is used herein to denote a molecule also having both hydrophobic and hydrophilic groups, but characterized by:
pKa: it has a pKa below 11.0, more preferably between about 11.0 and about 7.5;
Partition coefficient: in an n-octanol/buffer (aqueous phase) system it has a logD in the range between about −3.0 and about 2.5.

Without being limited to the above, the drugs may be any drug, the delivery of which via liposomes is desired.

In the context of the present disclosure it is required that the amphipathic drugs loaded into the liposomes are either weak acids or weak bases. In other words, both, in the case of two drugs, and all in the case of more than two drugs need to be either acids or bases in order to be effectively simultaneously loaded into liposomes.

In accordance with some embodiment, the drugs may be characterized by one or more of the following biochemical activities: antimetabolites, DNA damaging agent, topoisomerase I inhibitors, topoisomerase II inhibitors, alkylating agents, DNA synthesis inhibitors, apoptosis inducing agent, cell cycle inhibitor, anti-mitotic agents, anti-angiogenesis agent and anticancer antibiotics.

In some embodiments, the at least two amphipathic drugs are selected to provide a therapeutic effect by providing the same biochemical effect; in some other embodiments, the encapsulated drugs exhibit different mechanism of actions.

In one particular embodiment, the liposomes co-encapsulate two amphipathic drugs exhibiting two different mechanisms of action.

Examples of amphipathic drugs that may be co-encapsulated into the same liposome in accordance with the invention include, without being limited thereto,

Chemotherpeutics—anthracyclines, camptothecins, vincalkaloids, mitoxanthrone, bleomycin, ciprofloxacin, cytrabine, mitomycin, streptozocin, estramustine, mechlorethamine, melphalan, cyclophosphamide, triethylenethiophosphoramide, carmustine, lomustine, semustine, hydroxyurea, thioguanine, decarbazine, procarbazine, epirubicin, carcinomycin, N-acetyladriamycin, rubidazone, 5-imidodaunomycin, N-acetyldaunomycine, daunoryline;

It is noted that in the context of the invention, the preferred at least two drugs are chemotherapeutic anti cancer drugs.

In this connection, it is further noted that the at least two amphipathic drugs are not the combination of doxorubicin and Verapamil. Preferably the at least two amphipathic drugs do not comprise Verapamil.

Anti inflammatory drugs—methylprednisolone hemisuccinate, 1-methasone hemisuccinate;

Antioxidant—tempamine;

Anti anxiety muscle relaxants—diclofenac, pridinol;

Local anesthetics—lidocaine, bupivacaine, dibucaine, tetracaine, procaine;

Photosensitizers for photodynamic therapy—benzoporphyrin and its derivatives (e.g., visudyne);

Analgesics—opiods, non-steroidal anti-inflammatory drugs (NSAIDs);

Antimicrobial medications—pentamidine, azalides;

Antipsychotics—chlorpromazine, perphenazine;

The antiparkinson agents—budipine, prodipine, benztropine mesylate, trihexyphenidyl, L-DOPA, dopamine;

Antiprotozoals—quinacrine, chloroquine, amodiaquine, chloroguanide, primaquine, mefloquine, quinine;

Antihistamines—diphenhydramine, promethazine;

Antidepressants—serotonin, imipramine, amitriptyline, doxepin, desipramine;

Anti anaphylaxis agents—epinephrine;

Anticholinergic drugs—atropine, decyclomine, methixene, propantheline, physostigmine;

Antiarrhythmic agents—quinidine, propranolol, timolol, pindolol;

Fluorescent dyes—acridine orange, fluorescein, carboxyfluorescein;

Prostanoids—prostaglandins, thromboxane, prostacyclin;

Examples for combination of drugs in the context of the invention include, without being limited thereto, a camptothecin with vincalkaloid.

Camptothecin are Topoisomerase I inhibitors and include, without being limited thereto, irinotecan, topotecan, 9-amino camptothecin, 10,11-methylenedioxy camptothecin, 9-nitro camptothecin, TAS 103, 7-(4-methyl-piperazino-methylene)-10,11-ethylenedioxy-20(S)-camptothecin and 7-(2-N-isopropylamino)ethyl)-20(S)-camptothecin.

Vincaalkeloids are anti-mitotic and anti-microtubule agents. They are used as drugs in cancer therapy and as immunosuppressive drugs. These compounds are vinblastine, vincristine, vindesine and vinorelbine.

In one embodiment, the combination comprises the camptothecin-topotecan (TPT) and the vinca alkaloid-vincristine (VCR). This combination (TPT)-(VCR) is of particular interest at least for the following reasons:
The drugs act on cancer cells via different targets in the cell and affect different phases in the cell cycle: TPT converts the target, DNA topoisomerase I, into a cellular toxin leading to arrest in the S phase or G₂-M phase, while, VCR causes depolymerization of microtubules leading to mitotic arrest.
The dose-limiting toxicities of the two drugs are different; TPT has relatively few nonhematological side effects, while, VCR has peripheral neuropathy and does not cause myelosuppression.
TPT and VCR are both weak amphipathic amines (as shown in Table 1 below) and therefore, both can be remote loaded into the intra-liposome aqueous phase by using an intra liposome high/extra liposome medium low transmembrane gradient, such as ammonium sulfate gradient as described herein.
Furthermore, both TPT and VCR have established activity against the same pediatric solid tumors and act synergistically against colon cancer.

The liposomes according to the present invention also comprise a counter ion compatible with the two or more amphipathic drugs and with which the at least two weak amphipathic acid drugs or at least two weak amphipathic base drugs are to exchange location during incubation of the pre-formed liposomes with the buffered or un-buffered solution containing the drug.

As used herein, when referring to a counter ion compatible with the two or more amphipathic drugs, it is meant that the counter ion has very low or essentially no liposome membrane permeability via the liposome bilayer so as to be retained in the intraliposomal aqueous core during loading of the drug, and during storage. It has high solubility in the medium, and is capable of forming a salt with both drugs and does not reduce the activity of each drug. With respect to low permeability, for example, the permeability coefficient of Cl⁻ through a phospholipid bilayer is 7.6×10⁻¹ cm/s that of SO₄²⁻ and glucuronate⁻ is <10⁻¹² cm/s, while for dextran sulfate the permeability coefficient is approaching zero.

When the amphipathic drugs are weak amphipathic acids, the counter ion within the liposome is a cationic compound; when the amphipathic drugs are weak amphipathic bases, the counter ion within the liposome is an anionic compound.

Non-limiting examples of counter ions to be found in the liposome include:

Anionic (counter ion to quaternary amine or imine such as ammonium): sulfate, phosphate, citrate, glucuronate, chloride, borate, hydroxide, nitrate, cyanate, and bromide; as well as anionic polymers with which the ion is covalently linked to a polymer, and includes dextran sulfate, sucrose octasulfate, polyphosphate (triethylammonium salts) and carboxymethyl dextran.

Cationic (counter ion to a carboxylate such as formic acid, acetic acid, propanoic acid, butanoic acid) include calcium, magnesium, sodium and manganese.

In accordance with one embodiment of the invention, the counter ion is preferably sulfate, from ammonium sulfate.

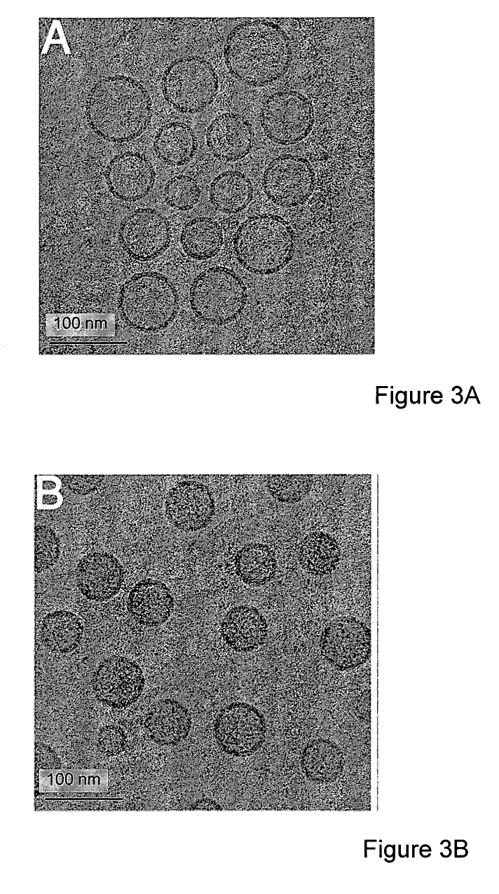

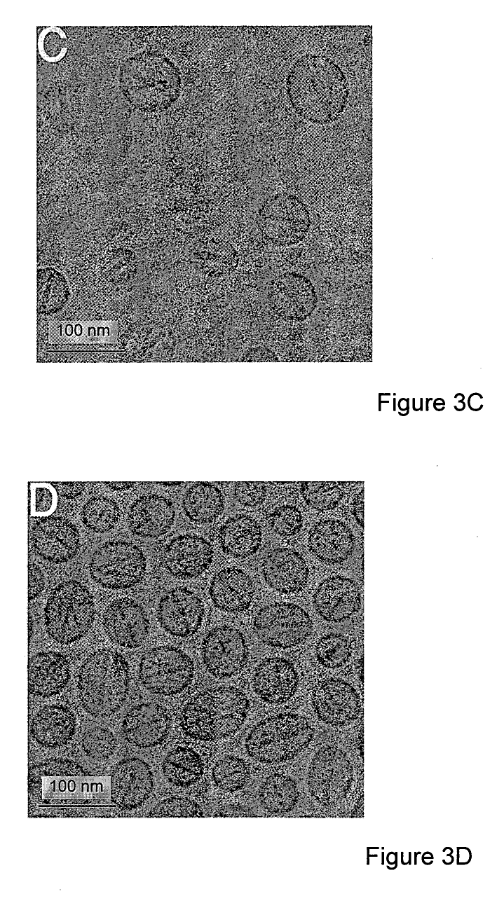

In some embodiments, at least a portion of the amphipathic drug in the intraliposomal aqueous core form a salt with the counter ion which precipitates in the aqueous phase; as also evident from the specific example provided hereinbelow. Specifically, FIG. 3B-3D showing precipitation of the drugs in the liposomes.

The ratio between the at least two amphipathic drugs in the intraliposomal aqueous core is pre-determined so as to achieve a desired therapeutic effect. In one embodiment, the pre-determined ratio between the at least two amphipathic drugs is the ratio between the maximal tolerated doses (MTD) of each amphipathic drug or is the synergistic molar ratio between the at least two amphipathic drugs.

When referring to “MTD” it is to be understood as encompassing the meaning known in the art, namely, the highest dose of a drug or drug combination that does not cause unacceptable side effects (toxicity). The MTD is determined in clinical trials by testing increasing doses on different groups of people until the highest dose with acceptable side effects is found. For each drug, the MTD as determined in clinical trials is then available via publicly available sources such as SciFinder which is the On Line search engine of The American Chemical Society for various information including MTD (in animals as well as in humans). When referring to MTD in cancer treatment, the MTD values may be defined as survival in the absence of significant tumor burden with ≦15% body weight loss nadir lasting ≦2 days. In this context, the “MTD ratio” between two drugs is the ratio between the MTD determined for each drug. For instance, when referring to topotecan (TPT, topotecan hydrochloride, MW 421.45) and vincristine (VCR, vincristine sulfate MW 923.04), being two amphipathic weak bases, the MTD is respectively, 5 mg/kg and 1.5 mg/kg, and the TPT/VCR mole ratio of 7.3.

When referring to “synergistic ratio” it is to be understood to encompassing the ratio at which the effect of the liposome comprising the at least two drugs is greater than the sum (additive) of effects of a mixture of two or more liposomes, each comprising a single drug. The synergistic ratio is determined by in vitro cytotoxicity of the at least two drugs and their combination, using, for example, the median-effect analysis of Chou et al. (T. C. Chou, P. Talaly, Quantitative analysis of dose-effect relationships: the combined effects of multiple drugs or enzyme inhibitors. Adv. Enzyme Regul. 22 (1984) 27-55; D. C. Rideout, T. C. Chou, Synergy, antagonism and potentiation in chemotherapy: An overview, Academic Press, San Diego, 1991), where the measure of synergy is defined by the Combination Index (CI) value. According to this method, drugs interact synergistically if CI is lower than 1.0, additively if CI is equal to 1.0, and antagonistically if CI is greater than 1.0, as a function of drug concentration for different fixed drug/drug mole ratios.

The liposomes of the invention exhibit a controlled release profile, where for at least a period of time the drugs are released from the liposomes at the pre-defined ratio, the period of time may be from an hour to a day and even to several days, and sufficient to achieve the simultaneous desired effect (preferably MTD effect) for both drugs at the target site.

In accordance with some preferred embodiments, the pre-determined ratio between the drugs is the ratio of MTD of the at least two amphapathic drugs to be simultaneously co-encapsulated in the liposome.

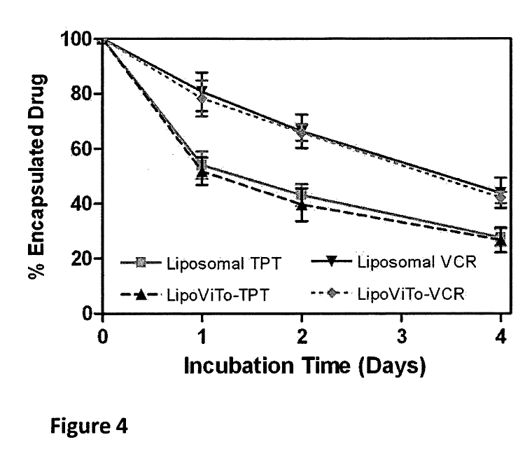

The drugs when co encapsulated in the liposomes also exhibit a liposomal profile that corresponds to the profile of each drug when encapsulated as a single drug in the same liposome. As used herein, the term “liposomal profile” is used to characterize physical parameters of the drug when in the liposome and these may include loading efficiency of the drug into the liposome, release profile of the drug from the liposome, of the drug from the liposome, solubility of the drug within the intraliposomal core, morphology of the drug within the intraliposomal core, etc. In the context of the invention, the liposomal profile of each drug, irrespective of whether encapsulated alone or with another amphipathic drug in a particular type of liposome (i.e. the same membrane composition comprising a single drug or a combination of drugs), will show substantial similarity in one or more of the above noted physical parameters. This characteristic of the liposomes of the invention is demonstrated, for example, in FIGS. 3 and 4 herein.

Further, in this context, the term “same liposome” denotes essentially the same or similar membrane composition and size of the liposome is used.

The liposomes of the invention are chemically as well as physically stable liposomes for a period of at least 3 months, and even for a period of 6 months during storage at 4° C. in a buffer, such as citrate buffer. The “stability” in the context of the present disclosure may be determined by the following methods:

Chemical stability can be determined by measuring, for example, liposome change/decrease in pH, or phospholipid (PL) acylester hydrolysis (by determining level of non-esterified (free) fatty acids (NEFA) released during storage due to the PL hydrolysis. Thus, for instance, if after a determined time in storage there is no significant change in level of pH (+0.5) or in level of NEFA (e.g. less than 5%), it can be concluded that the liposome is chemically stable. In addition, chemical stability may be determined using High performance liquid chromatography (HPLC).

Physical stability can be determined by liposome size distribution using dynamic light-scattering (DLS), cryo transmission electron microscopy, or level of free (non-liposome) material (e.g. drug) being sequestered out of the liposome, by separating (e.g. by centrifugation, gel permeation chromatography, ion exchange chromatography or gradient centrifugation) the liposomes from nondispersable matter and analyzing by HPLC or TLC, (using for example silica gel plates) free (non liposome associated material composition while liposome concentration is determined by as phospholipid content determined as organic phosphorus by the Bartlett method, or by HPLC.

As shown in the exemplary embodiments, the liposomes co-encapsulating two amphipathic drugs were chemically as well as physically stable for a period of at least 6 months, during storage at 4° C. in a buffer medium. Further, drugs release and size distribution changes during six months were below detection limits.

In accordance with some embodiments, the stability of the liposomes of the present disclosure, encapsulating at least two drugs, is exhibited by a drug concentration of at least 85%, at least 90% and even at least 95% of the maximal solubility of the drug in water, for each encapsulated drug in the intraliposomal aqueous core during storage for a long period of time, such as for at least 6 months.

The liposomes co-encapsulating two amphipathic drugs according to the present disclosure exhibit a controlled release profile of the drugs, with the predetermined ratio being maintained following administration. In one embodiment, the drug release profiles from liposomes loaded with individual drugs are essentially the same as the release rates from the 2 drugs co-encapsulating liposome.

In one embodiment, the release of the two drugs is simultaneous. This is in line with reports that cancer cells take up nanoliposomes. Thus, co-encapsulating two or more drugs in one liposome assures that the cancer cell is “attacked” by both drugs simultaneously, while treatment with a mixture of liposomes might result in heterogenous exposure of the cells to both drugs, e.g. 15% of the tumor cells being exposed to a first drug, 15% being exposed to a second drug and 70% being exposed to both drugs.

Co-encapsulation of at least two drugs in the liposome also permits a reduced total dose of injected lipid as compared to administration of individually loaded liposomes and also reduces risks of having one liposome population affecting the pharmacokinetic profile of the other, thereby altering drug delivery, when two or more liposomes populations are administered.

The at least two amphipathic drugs are simultaneously loaded, by the same method, into pre-formed liposomes and the present disclosure also provides a method for the simultaneous co-enacpsulation into a liposome of the at least two amphipathic drugs.

In accordance with the present disclosure, the method comprises:
providing a suspension of liposomes comprising in the intraliposomal aqueous core of the liposome a weak acid or weak base and a counter ion of the weak acid or weak base, the concentration of the weak acid or weak base being greater inside the liposome than outside the liposome;
simultaneously incubating the liposomes with at least two amphipathic drugs having a pre-determined ratio therebetween, the at least two amphipathic drugs being compatible with the counter ion, wherein, when the liposomes comprise a weak acid, the at least two amphipathic drugs are weak amphipathic acid drugs, and when the liposomes comprise a weak base, the at least two amphipathic drugs are weak amphipathic base drugs;

wherein,
the liposome comprises one or combination of liposome forming lipids, the one or combination of liposome forming lipids have a solid ordered (SO) to liquid disordered (LD) phase transition temperature above 37° C.;
the incubation is under conditions sufficient to allow simultaneous co-encapsulation in the intraliposomal aqueous core of the liposome of the two amphipathic drugs without use of a transition metal and/or a ionophore and the encapsulation is at a pre-determined ratio between the at least two amphipathic drugs;
when in the liposome, each of the at least two amphipathic drugs exhibit a liposomal profile that corresponds to the profile of each drug when encapsulated as a single drug in the same liposome; and
for each drug, the method provides a loading efficiency above 85%.

The loading of the at least two amphipathic drugs does not require the complexation with a chelating agent, e.g. transition metal ion such as Mn (the chelating agent being Mn-sulfate) or the use of ionophores, as required by other methods for co-encapsulation of two drugs into liposomes, such as that described for the loading of VCR and doxorubicin.

The liposomes are pre-formed liposomes. Liposomes can be formed by various techniques, as well known in the art, such as hydration of a lipid film/cake, reverse-phase evaporation and solvent infusion. The thus formed liposomes may then be sized by techniques known in the art, as also discussed above.

The pre-formed liposomes are then treated to exhibit a pH or ion gradient with respect to its surrounding, also known by the term “remote loading” or “active loading”. After sizing, the external medium of the liposomes is treated to produce an ion gradient across the liposome membrane (e.g. with the same buffer used to form the liposomes) the gradient being a higher inside/lower outside ion concentration gradient. This may be done in a variety of ways, e.g., by (i) diluting the external medium, (ii) dialysis against the desired final medium, (iii) gel exclusion chromatography, e.g., using Sephadex G-50, equilibrated in the desired medium which is used for elution, or (iv) repeated high-speed centrifugation and resuspension of pelleted liposomes in the desired final medium. The external medium which is selected will depend on the type of gradient, on the mechanism of gradient formation and the external solute and pH desired.

In the simplest approach for generating an ion and/or H⁺ gradient, the lipid film/cake is hydrated and sized in a medium having a selected internal-medium pH. The suspension of the liposomes is titrated until the external liposome mixture reaches the desired final pH, or treated to exchange the external phase buffer with one having the desired external pH. For example, the original hydration medium may have a pH of 5.5, in a selected low permeability buffer, e.g., glutamate, citrate, succinate, fumarate buffer, and the final external medium may have a pH of 8.5 in the same or different buffer. The common characteristic of these buffers is that they are formed from acids which are essentially liposome impermeable. The internal and external media are preferably selected to contain about the same osmolarity, e.g., by suitable adjustment of the concentration of buffer, salt, or low molecular weight non-electrolyte solute, such as dextrose or sucrose.

In one embodiment, the proton gradient used for drug loading is produced by creating an ammonium gradient across the liposome membrane, as described, for example, in U.S. Pat. Nos. 5,192,549 and 5,316,771. The liposomes are prepared in an aqueous buffer containing the ammonium salt, such as ammonium sulfate, ammonium phosphate, ammonium citrate, etc., typically 0.1 to 0.3 M ammonium salt, at a suitable pH, e.g., 5.5 to 7.5.

As already mentioned above, the gradient can also be produced by including in the hydration medium polymers to which the counter ion is covalently attached. Such charged polymers sulfated polymers, such as dextran sulfate ammonium salt, heparin sulfate ammonium salt or sucralfate.

After liposome formation and sizing, the external medium is exchanged for one lacking ammonium ions. In this approach, during the loading the amphipathic weak base is exchanged with the ammonium ion. The same approach may be used for loading amphipathic weak acids, with the salt containing a weak acid to exchange with the drug. Accordingly, as also described in U.S. Pat. No. 5,939,096, the method employs a proton shuttle mechanism involving the salt of a weak acid, such as acetic acid, of which the protonated form trans-locates across the liposome membrane to generate a higher inside/lower outside pH gradient. The amphipathic weak acid may then be added to the medium to the pre-formed liposomes. This amphipathic weak acid accumulates in liposomes in response to this gradient, and may be retained in the liposomes either by cation (i.e. calcium ions)-promoted precipitation or low permeability across the liposome membrane, namely, the amphipathic weak acid is exchanges with the acetic acid.

The at least two amphipathic drugs may be added in the medium comprising the liposomes in dry form (e.g. powder) or in solution, prior to incubation with the suspension of liposomes. It is essential however that once in incubation, the drug is at least partially in soluble form and at least part thereof is in uncharged form. The concentration of the drugs prior to incubation is set according to pre-determined values, based on the desired loading concentrations.

The at least two amphipathic drugs are then incubated with the liposome suspension under conditions that support simultaneous remote loading of the drugs into the liposomes. The conditions may include temperature, typically between 25° C. to 70° C., at times, between 45° C.-70° C. and time, for several minutes or more, as known for remote loading.

In a preferred embodiment, the loading of the at least two amphipathic drugs is against an ammonium salt gradient.

It has been found that the loading of the two drugs when using remote loading against the same driving force, e.g. ammonium salt, is high, at an efficiency similar to that of each drug when encapsulated alone, or above 85%, or even 90%, and at times even above 95% or even above 98%, determined according to the formula below (PL being the phospholipid):

$$Loading efficiency = 100 \times \frac{{( [ drug ] / [ PL ] )}_{after loading}}{( {[ drug / [ PL ] )}_{before loding}}$$

This high loading efficiency allows maintenance of the initial drug ratio, i.e. initial drug ratio (prior to loading)≈final drug ratio in the liposome. In other words, the high loading efficiency ensures pre-determining the concentration of encapsulated drugs and drug ratios, by controlling the initial drug ratio in the system prior to loading.

The invention also provides a pharmaceutical composition comprising a physiologically acceptable carrier and liposomes co-encapsulating at least two amphipathic drugs, as disclosed herein; as well as a method of treatment of a subject comprising administering to the subject the liposomes disclosed herein, typically in the form of a pharmaceutical composition comprising the liposomes and the physiologically acceptable carrier.

The liposomes in combination with physiologically acceptable additives and carriers may be administered by any route acceptable in the art.

According to one embodiment, the administration of the composition of matter is in a form suitable for systemic delivery of the drugs, e.g. by injection or infusion or other means for parenteral administration. This includes, without being limited thereto, intravenous (i.v.), intraarterial (i.a.), intramuscular (i.m.), intracerebral, intracerebroventricular, intracardiac, subcutaneous (s.c.), intraosseous (into the bone marrow), intradermal, intratheacal, intraperitoneal, intravesical, and intracavernosal and epiduaral (peridural) injection or infusion.

Parenteral administration for systemic delivery may also include transdermal, e.g. by transdermal patches, transmucosal (e.g. by diffusion or injection into the peritoneum), inhalation and intravitreal (through the eye).

A preferred mode of administration is injection, more preferably intravenous (i.v.) injection. The requirements for effective pharmaceutical vehicles for injectable formulations are well known to those of ordinary skill in the art (see for example Pharmaceutics and Pharmacy Practice, J.B. Lippincott Co., Philadelphia, Pa., Banker Chalmers, Eds., pages 238-250 (1982), and ASHP Handbook on Injectable Drugs, Toissel, 12^{th} Ed. (2002)).

“Treatment” in the context of the invention denotes any therapeutic effect achieve by the administration of the liposomes to a subject in need thereof, which may include alleviating a pathological condition for which at least one of the weak amphipathic drug is known to be effective, or at least alleviating one of its undesired side effect. Treatment also encompass reducing severity of a pathological condition or duration of its acute phase or cure altogether, slowing down deterioration of symptoms of a pathological condition; slowing down the progression of a pathological condition; enhance onset of remission periods of a pathological condition, slowing down or prevent any irreversible damage caused by a pathological condition, lessening the severity of the pathological condition, improving survival rate and more rapid recovery from the pathological condition or preventing the condition from occurring or any combination of the above.

For example, the pathological condition for which at least one of the weak amphipathic drug is known to be effective is abnormal proliferation of cells, such as in cancer. To this end, treatment denotes, inter alia, inhibition or reduction of the growth and proliferation of tumor cells: including arresting growth of the primary tumor, or decreasing the rate of cancer related mortality, or delaying cancer related mortality, which may result in the reduction of tumor size or total elimination thereof from the individual's body, or decreasing the rate of occurrence of metastatic tumors, or decreasing the number of metastatic tumors appearing in an individual, inhibition of organization of cells such as neo-vascularization.

Further, for example, the pathological condition for which at least one of the weak amphipathic drug is known to be effective is a neurodegenertive condition, which includes any abnormal deterioration of the nervous system resulting in the dysfunction of the system, including relentlessly progressive wasting away of structural elements of the nervous system exhibited by any parameter related decrease in neuronal function, e.g. a reduction in mobility, a reduction in vocalization, decrease in cognitive function (notably learning and memory) abnormal limb-clasping reflex, retinal atrophy inability to succeed in a hang test, an increased level of MMP-2, an increased level of neurofibrillary tangles, increased tau phosphorylation, tau filament formation, abnormal neuronal morphology, lysosomal abnormalities, neuronal degeneration, gliosis and demyelination. In this context, treatment includes administration to prevent, inhibit or slow down abnormal deterioration of the nervous system, to ameliorate symptoms associated with a neurodegenerative condition, to prevent the manifestation of such symptoms before they occur, to slow down the irreversible damage caused by the chronic stage of the neurodegenerative condition, to lessen the severity or cure a neurodegenerative condition, to improve survival rate or more rapid recovery form neurodegeneration.

For the purpose of effective delivery, the liposomes are formulated to provide an effective amount of the two drugs. The effective amount in the composition is dictated by the pre-determined synergetic mole ratio or MTD ratio.

The liposome containing composition may provided as a single dose or as multiple doses administered to the subject over a period or time (e.g. to produce a cumulative effective amount) in a single daily dose, in several doses a day, as a single dose for several days, etc. The treatment regimen and the specific formulation to be administered will depend on the type of disease to be treated and may be determined by various considerations, known to those skilled in the art of medicine, e.g. the physicians.

Further provided by the invention is a package (pharmaceutical kit) comprising a liposomes as disclosed herein and instructions for administration of the liposomes to a subject in need thereof. The package may include lyophilized liposomes comprising the co-encapsulated drugs or ready to use composition, where the liposomes with the at least two drugs encapsulated therein are in suspended form. The package may also include means for administration of the composition, such as a syringe.

Yet further, the invention provides the use of liposomes as disclosed herein, for the preparation of a pharmaceutical composition for the treatment of a condition for which at least one of the weak amphipathic drug is known to be effective; as well as liposomes as disclosed herein for the treatment of a condition for which at least one of the weak amphipathic drug is known to be effective.

### DESCRIPTION OF SOME NON-LIMITING EXAMPLES

### Materials and Methods

### Chemicals:

Vincristine (VCR) sulfate (Avachem Scientific, San Antonio, Tex.)
Topotecan (TPT) hydrochloride, (Sinova, Bethesda, Md.)
Radiolabeled vincristine sulfate [³H], (ARC, St. Louis, Mo.).
Phospholipon® 100 H (hydrogenated soybean phosphatidyl choline (HSPC), Tₘ 55° C.) (Phospholipid, Hermesberg, Germany).
Cholesterol (Sigma, St. Louis, Mo.);
1,2-distearoyl-sn-glycero-3-phosphoethanolamine-N-[methoxy(polyethylene glycol)-2000](^{2k}PEG-DSPE) (Genzyme Pharmaceuticals (Liestal, Switzerland).
Cholesterol hexadecyl ether (CHE) radiolabelled with [¹⁴C](ARC, St. Louis, Mo.).

### Cells:

Daoy human medulloblastoma cell line and SW480 human colon cancer (American Type Culture Collection, Manassas, Va.).
NB-EB neuroblastoma tumor cells (from Peter J. Houghton, St. Jude Children's Research Hospital, Memphis, Tenn., (P. J. Houghton, P. J. Cheshire, L. Myers, C. F. Stewart, T. W. Synold, J. A. Houghton, Evaluation of 9-dimethylaminomethyl-10-hydroxycamptothecin against xenografts derived from adult and childhood solid tumors. Cancer Chemother Pharmacol 31(3) (1992) 229-239).

### Animals:

Five weeks old NUDE-Hsd:Athymic mice (Harlan Laboratories, Jerusalem, Israel).

### Cell Culture:

Human medulloblastoma (Daoy) cells, colon cancer (SW480) cells and neuroblastoma (NB-EB) cells were exposed to fixed ratios at eight concentrations of drugs for 72 hours along the profile of the most cytotoxic drug. Viable cells were quantified using standard MTT assay (T. Mosmann, Rapid colorimetric assay for cellular growth and survival: application to proliferation and cytotoxicity assays. J Immunol Methods 65(1-2) (1983) 55-63).

### Proliferation Data Analysis:

Test data were converted to a percentage mean cell survival value relative to untreated control wells. The fraction of affected cells (fₐ) was subsequently determined for each well. Three replicates were averaged and three repeats of these data sets were analyzed by the median effect analysis (T. C. Chou, P. Talaly, Quantitative analysis of dose-effect relationships: the combined effects of multiple drugs or enzyme inhibitors. Adv. Enzyme Regul. 22 (1984) 27-55; D. C. Rideout, T. C. Chou, Synergy, antagonism and potentiation in chemotherapy: An overview, Academic Press, San Diego, 1991).

The median effect analysis uses the combination index (CI) value as a quantitative indicator of the degree of synergy or antagonism. Using this analysis method, CI=1.0 reflects additive activity, CI>1 signifies antagonism, and CI<1.0 indicates synergy.

### Lipids:

Phospholipon®100 H (HSPC, Tₘ 55° C.) has an iodine value of 1.0, ˜85% stearic acid (C18:0), ˜15% palmitic acid (C16:0), and <1% other acyl chains. Phospholipid concentration was determined using a modified Bartlett procedure (H. Shmeeda, S. Even-Chen, R. Honen, R. Cohen, C. Weintraub, Y. Barenholz, Enzymatic assays for quality control and pharmacokinetics of liposome formulations: comparison with nonenzymatic conventional methodologies. Methods Enzymol. 367 (2003) 272-292) and ¹⁴C CHE liquid scintillation counting.

### Preparation of Liposomes:

Nanoliposomes composed of the HSPC, cholesterol, and ^{2k}PEG-DSPE (54:41:5 mole ratio) were prepared as previously described (D. Zucker, D. et al. Marcus, Y. Barenholz, A. Goldblum, Liposome Drugs' Loading Efficiency: A Working Model Based on Loading Conditions and Drug's Physicochemical Properties. J. Control. Release 139 (2009) 73-80). In short, first a mixture of the desired PC (in most cases HSPC), cholesterol, and ^{2k}PEGDSPE (54:41:5 mole ratio) in ammonium sulfate to form multilamellar vesicles (MLV) by the ethanol injection method. These MLV were downsized to large unilamellar vesicles (LUV; 100±20 nm), by medium-pressure stepwise extrusion through polycarbonate filters (400 to 100 nm pore size) using the Northern Lipids, Inc. (Burnaby, BC, Canada) extruder device. Small unilamellar vesicles (SUV; 80±15 nm), were then formed by an additional extrusion step using a 50 nm pore size polycarbonate filter.

nSSL Characterization:

The nSSL were characterized for their ζ-potential and size distribution by Malvern's Zetasizer Nano ZS instrument (Worcestershire, United Kingdom). These were −6.6±2.9 mV and 120±10 nm, respectively for all formulations in dextrose 5% medium.

Membrane “fluidity” of the liposomes was determined by fluorescence anisotropy of the fluorophore 1,6-diphenyl-1,3,5-hexatriene (DPH) (M. Shinitzky, Y. Barenholz, Dynamics of the Hydrocarbon Layer in Liposomes of Lecithin and Sphingomyelin Containing Dicetylphosphate. Journal of Biological Chemistry 249(8) (1974) 2652-2657; M. Shinitzky, Y. Barenholz, Fluidity parameters of lipid regions determined by fluorescence polarization. Biochim Biophys Acta 515(4) (1978) 367-394). The DPH was added to the liposomes formulation (final mole ratio of total lipid to probe was 400:1), followed by 30 min incubation in the dark at 37° C. to achieve complete insertion of the DPH into the hydrophobic region of the liposome bilayer (M. Shinitzky, Y. Barenholz, Dynamics of the Hydrocarbon Layer in Liposomes of Lecithin and Sphingomyelin Containing Dicetylphosphate. Journal of Biological Chemistry 249(8) (1974) 2652-2657; M. Shinitzky, Y. Barenholz, Fluidity parameters of lipid regions determined by fluorescence polarization. Biochim Biophys Acta 515(4) (1978) 367-394; V. Borenstain, Y. Barenholz, Characterization of liposomes and other lipid assemblies by multiprobe fluorescence polarization. Chem Phys Lipids 64(1-3) (1993) 117-127).

The degree of DPH anisotropy

$$( r = \frac{{I}_{II} - {I}_{⊥}}{{I}_{II} + 2 {I}_{⊥}} )$$

in the labeled liposomes in PBS was calculated from the fluorescence intensity at the parallel (I_{II}) and perpendicular (I_{⊥}) planes, using the Synergy 4 fluorescent plate reader (BioTek, USA), at excitation/emission wavelengths of 360/430 nm. Anisotropy values of 0.372±0.0008 (25° C.), 0.371±0.0007 (37° C.) and 0.366±0.0004 (50° C.) were measured. The maximal anisotropy value is 0.4; therefore these liposomes are highly rigid. The reason is that at all temperature of the measurements were below HSPC's Tₘ (55° C.).

### Trans-Membrane Ion Gradient Formation:

The salt in the external liposome medium was replaced by dialysis as previously described [D. Zucker et al. 2009, ibid.].

### Drug Encapsulation:

The commercially available topotecan (TPT) and vincristine (VCR), both weak amphipathic anticancer drugs, were mixed, as are, with the preformed nSSL dispersion exhibiting a transmembrane ammonium salt gradient. The remote loading was achieved by incubation of the liposomes with the drugs for 30 min at 55° C., then cooling to 4° C., followed by dialyzing against 5% dextrose to remove ammonia and residual unloaded drug. Alternatively, in some cases, unloaded drug and ammonia (released during the loading process) were removed using cation exchange resin Dowex 50WX-4 (G. Haran, R. Cohen, L. K. Bar, Y. Barenholz, Transmembrane ammonium sulfate gradients in liposomes produce efficient and stable entrapment of amphipathic weak bases. Biochim. Biophys. Acta 1151(2) (1993) 201-215; G. Storm, L. van Bloois, M. Brouwer, D. J. Crommelin, The interaction of cytostatic drugs with adsorbents in aqueous media. The potential implications for liposome preparation. Biochim. Biophys. Acta 818(3) (1985) 343-351). At times, the co-encapsulated nSSL with VCR and TPT is referred to by the abbreviation “LipoViTo”.

### Cryo-Transmission Electron Microscopy (TEM):

Cryo-TEM was used to confirm liposome size distribution measured by dynamic light scattering and to characterize the detailed structure of the nSSLs, as previously described [A. Schroeder, Y. Avnir, S. Weisman, Y. Najajreh, A. Gabizon, Y. Talmon, J. Kost, Y. Barenholz, Controlling liposomal drug release with low frequency ultrasound: mechanism and feasibility. Langmuir 23(7) (2007) 4019-4025). Briefly, Cryo-TEM work was performed at Oren Regev's Laboratory (Ben Gurion University, Beer Sheva, Israel). For each experiment, lipid dispersions at concentrations of 50 and 5 mM in 5% (w/v) dextrose in a total volume of 400 μL were used. Specimens were prepared in a controlled-environment vitrification system at 25° C. and 100% relative humidity and then examined in a Philips CM120 cryo-electron microscope operated at 120 kV. Specimens were equilibrated in the microscope below −178° C., examined in the low-dose imaging mode to minimize electron beam radiation damage, and then recorded at a nominal underfocus of 4-7 nm to enhance phase contrast.50 An Oxford CT-3500 cooling holder was used. Images were recorded digitally with a Gatan MultiScan 791 CCD camera using the Digital Micrograph 3.1 software package.

### Drug Quantification:

Quantification was performed using HPLC with UV and fluorescence detectors for VCR and TPT, respectively, as described by Zucker et al. (D. Zucker, et al. 2009 ibid.).

Briefly, the system included Kontron 420 HPLC pump, Kontron HPLC 460 autosampler and Kontron 450 data system (Switzerland). TPT was quantified using a Waters Symmetry C18 column (150 mm×4.6 mm, 5 μm) with a fluorescence detector (Jasco Model FP-210) at excitation/emission wavelengths of 416/522 nm. Mobile phase A consisted of water, acetic acid, and triethylamine (97.9:0.6:1.5, v/v/v) and mobile phase B of water, acetic acid, triethylamine, and acetonitrile (57.9:0.6:1.5:40 v/v/v/v). The separation consisted of a gradient method, beginning at 33.8% of mobile phase A for 5 min and increasing to 100% (from the 5^{th} min to the 9^{th}). At these conditions the carboxylate form of TPT elutes after ˜4 min and the lactone after ˜7 min. Vincristine was quantified using an ACE C18 column (150 mm×4.6 mm, 5 μm) with UV detector (Kontron, Model 430) at 221 nm; Samples were eluted with mixture of phosphate buffer 0.04 M, pH 3 and methanol. The separation consisted of a gradient method, beginning at 30% methanol and increasing to 70% methanol. For both drugs, flow speed was 1.0 ml/min and injection volume was 20 μl.

In Vitro Release of Drugs from Nanoliposomes:

For studying the effect of biological fluids, drug loaded nSSLs were incubated up to 96 h at 37° C. in adult bovine serum (Biological Industries, Beit Haemek, Israel). Aliquots were taken from the incubated liposomes at the desired time points, and the released drugs were efficiently removed from the drug loaded nSSL by the cation exchange resin Dowex 50WX-4. ¹⁴C CHE (cholesteryl ether) Liposomes and ³H vincristine concentrations were determined by liquid scintillation counting, while TPT concentrations were determined by HPLC equipped with a fluorescence detector.

### Efficacy Evaluations:

Approximately 4 million Daoy and SW480 tumor cells were inoculated subcutaneously (s.c.) in the back of 5 weeks old NUDE-Hsd:Athymic mice. Tumor weights were determined according to the equation (length×width²)/2 using direct caliper measurements (D. M. Euhus, C. Hudd, M. C. LaRegina, F. E. Johnson, Tumor measurement in the nude mouse. J Surg Oncol 31(4) (1986) 229-234; M. M. Tomayko, C. P. Reynolds, Determination of subcutaneous tumor size in athymic (nude) mice. Cancer Chemother Pharmacol 24(3) (1989) 148-154).

Maximum tolerated dose (MTD) values were defined as survival in the absence of significant tumor burden with ≦15% body weight loss nadir lasting ≦2 days. Tumor volume, survival, and body weight were monitored 2-3 times per week.

The statistical significance between different treatment groups was determined using Mood's median test [A. M. Mood, Introduction to the theory of statistics, McGraw-Hill, New York, 1950.].

### Results:

### In Vitro Screening of VCR and TPT for Synergy

Firstly, in vitro cytotoxicity of VCR, TPT and their combinations was evaluated using the median-effect analysis of Chou et al [T. C. Chou, P. Talaly, Quantitative analysis of dose-effect relationships: the combined effects of multiple drugs or enzyme inhibitors. Adv. Enzyme Regul. 22 (1984) 27-55; D. C. Rideout, T. C. Chou, Synergy, antagonism and potentiation in chemotherapy: An overview, Academic Press, San Diego, 1991], where the measure of synergy is defined by the CI value. The selection of this drug interaction analysis method was based on its suitability for assessing whether drugs interact synergistically (CI<1.0), additively (CI f 1.0), or antagonistically (CI>1.0) as a function of drug concentration for different fixed drug/drug ratios.

Firstly the fraction of killed cells was measured at various drug concentrations (Table 1).

TABLE 1
Toxicity of vincristine (VCR) and topotecan (TPT) to Daoy
medulloblastoma cells

| Compound | Dose | Fractional | log | Log | |
|---|---|---|---|---|---|
| or mixture | (ng/ml) | kill (fₐ) | (dose) | [1/(1/fₐ − 1)] | Parameters |
| Vincristine | 1 | 0.04 | 0.00 | −1.43 | r: 0.982 |
| (VCR) | 2 | 0.11 | 0.30 | −0.93 | m: 1.926 |
| | 4 | 0.30 | 0.60 | −0.36 | b: −1.43 |
| | 8 | 0.82 | 0.90 | 0.65 | Dₘ: 5.505 |
| | 24 | 0.92 | 1.38 | 1.07 | |
| Topotecan | 40 | 0.18 | 1.60 | −0.64 | r: 0.988 |
| (TPT) | 60 | 0.46 | 1.78 | −0.08 | m: 1.663 |
| | 80 | 0.52 | 1.90 | 0.04 | b: −3.18 |
| | 200 | 0.80 | 2.30 | 0.59 | Dₘ: 81.23 |
| | 400 | 0.93 | 2.60 | 1.15 | |
| TPT:VCR | 35 | 0.00 | 1.54 | −3.00 | r: 0.973 |
| (73.1) | 70 | 0.06 | 1.85 | −1.19 | m: 6.277 |
| | 140 | 0.27 | 2.15 | −0.43 | b: −13.01 |
| | 200 | 0.93 | 2.30 | 1.12 | Dₘ: 117.990 |
| | 400 | 1.00 | 2.60 | 4.00 | |
| TPT:VCR | 25 | 0.03 | 1.40 | −1.51 | r: 0.947 |
| (2.9:1) | 50 | 0.13 | 1.70 | −0.83 | m: 4.829 |
| | 100 | 0.67 | 2.00 | 0.31 | b: −8.78 |
| | 200 | 0.98 | 2.30 | 1.69 | Dₘ: 65.667 |
| | 280 | 1.00 | 2.45 | 4.00 | |

r in Table 1 is the correlation coefficient,
m is the slope (Hill-type coefficient signifinying the sigmoidicity of the dose-effect curve) and
b is the Y-axis interscept of the tredline, and
Dₘ is the dose required to produce the median-effect.

These parameters were calculated by the following formulas:

$$r = \frac{n \sum ( x y ) - \sum x \sum y}{\sqrt{[ n \sum ( {x}^{2} ) - {( \sum x )}^{2} ] [ n \sum ( {y}^{2} ) - {( \sum y )}^{2} ]}}$$

$$m = \frac{n \sum ( x y ) - \sum x \sum y}{n \sum ( {x}^{2} ) - {( \sum x )}^{2}}$$

$$b = \frac{\sum y - m \sum x}{n}$$

The limits of the summation, which are i to n, and the summation indices on x and y have been omitted.

Dₘ=10^{(-b/m)}

At each given effect level (fₐ) the doses Dₓ₁, Dₓ₂ and Dₓ₁₂ were calculated with the use of the following equation:

$$D = {{D}_{m} [ \frac{{f}_{a}}{1 - {f}_{a}} ]}^{1 / m}$$

The contribution of D₁ and D₂ in the mixture Dₓ₁₂ was calculated from the known dose ratio of the two drugs. For example, if D₁/D₂=p/q, then:

$${D}_{1} = {D}_{x 12} \times \frac{p}{p + q}$$

$${D}_{2} = {D}_{x 12} \times \frac{p}{p + q}$$

The combination index (CI) was calculated by the following equation:

$$C I = \frac{{D}_{1}}{{D}_{x 1}} + \frac{{D}_{2}}{{D}_{x 12}} + \frac{{D}_{1} {D}_{2}}{{D}_{x 1} {D}_{x 2}}$$

TABLE 2
Calculated Dₓ₁, Dₓ₂, Dₓ₁₂, D₁, D₂ and CI based on the data in Table 1.

| | VCR | TPT | TPT:VCR (73:1) | | | | TPT:VCR (2.9:1) | | | |
|---|---|---|---|---|---|---|---|---|---|---|
| fₐ | Dₓ₁ | Dₓ₂ | Dₓ₁₂ | D₁ | D₂ | Cl | Dₓ₁₂ | D₁ | D₂ | Cl |
| 0.1 | 1.76 | 21.67 | 83.14 | 1.12 | 82.02 | 6.84 | 43.24 | 11.09 | 32.15 | 0.90 |
| 0.2 | 2.68 | 35.29 | 94.61 | 1.28 | 93.33 | 4.38 | 47.81 | 12.26 | 35.55 | 0.72 |
| 0.3 | 3.55 | 48.80 | 103.09 | 1.39 | 101.70 | 3.30 | 51.12 | 13.11 | 38.01 | 0.64 |
| 0.4 | 4.46 | 63.65 | 110.61 | 1.49 | 109.12 | 2.62 | 54.00 | 13.85 | 40.15 | 0.58 |
| 0.5 | 5.50 | 81.23 | 117.99 | 1.59 | 116.40 | 2.14 | 56.78 | 14.56 | 42.22 | 0.54 |
| 0.6 | 6.79 | 103.66 | 125.86 | 1.70 | 124.16 | 1.75 | 59.71 | 15.31 | 44.40 | 0.50 |
| 0.7 | 8.55 | 135.22 | 135.04 | 1.82 | 133.22 | 1.41 | 63.07 | 16.17 | 46.90 | 0.47 |
| 0.8 | 11.31 | 186.99 | 147.15 | 1.99 | 145.16 | 1.09 | 67.43 | 17.29 | 50.14 | 0.43 |
| 0.9 | 17.22 | 304.53 | 167.44 | 2.26 | 165.18 | 0.75 | 74.56 | 19.12 | 55.44 | 0.39 |

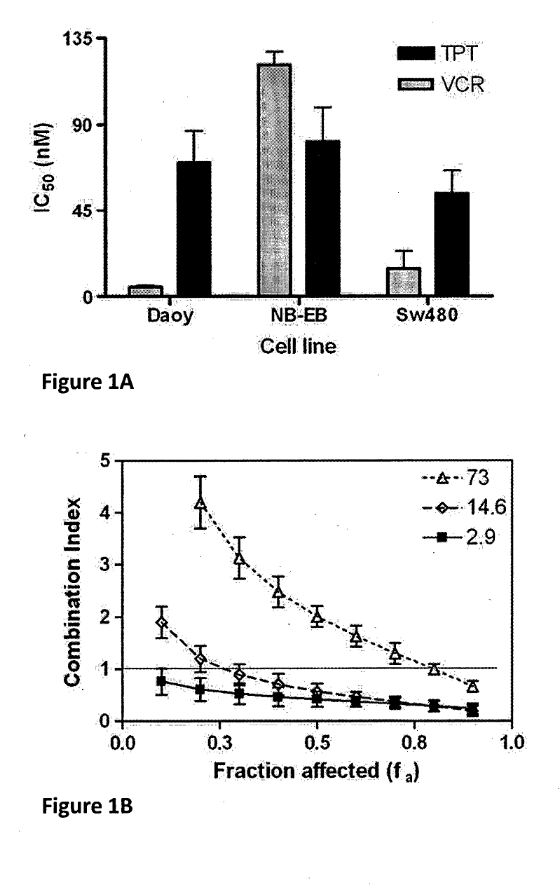

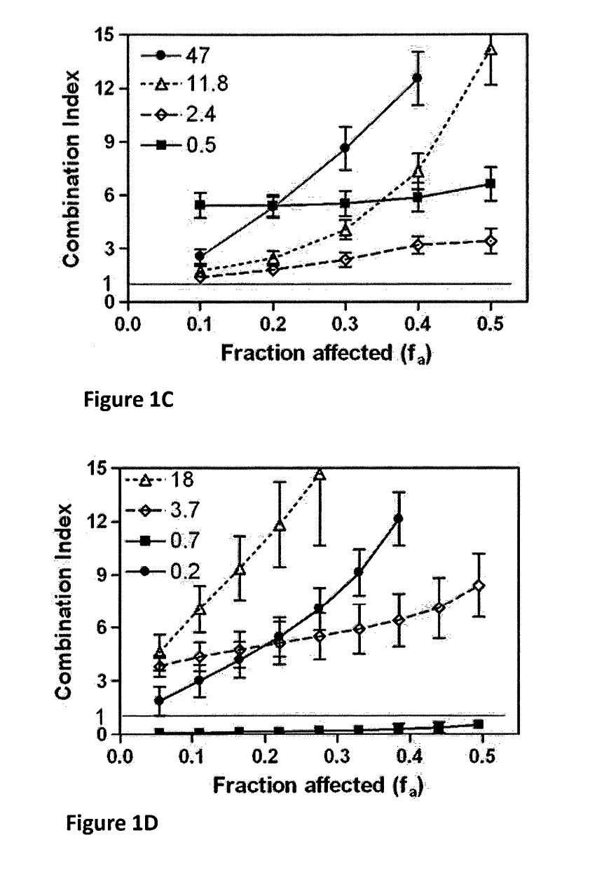

Cytotoxicity study was conducted by arbitrarily varying drug concentrations for each drug to estimate the potency of the drugs. VCR was found to be more potent than TPT by 15 fold and by 4 fold in, respectively, Daoy and SW480 cells, (FIG. 1A).

Therefore, it was decided that an equipotent mixture of TPT and VCR required a mole ratio of TPT/VCR>1. FIGS. 1B-1D summarize the results of the cytotoxicity analysis (Combination Index) done by exposing human medulloblastoma (Daoy), neuroblastoma (NB-EB) and SW480 colon cancer cells to various ratios and concentrations of VCR and TPT. Synergistic interactions were observed in vitro at certain drug/drug mole ratio ranges, whereas other ratios resulted in an additive or antagonistic effect. This finding is in line with previous teachings that the combination of vincristine and topotecan interact synergistically in vitro under appropriate conditions (J. Thompson, E. O. George, C. A. Poquette, P. J. Cheshire, L. B. Richmond, S. S. de Graaf, M. Ma, C. F. Stewart, P. J. Houghton, Synergy oftopotecan in combination with vincristine for treatment of pediatric solid tumor xenografts. Clin. Cancer Res. 5(11) (1999) 3617-3631; H. R. Bahadori, M. R. Green, C. V. Catapano, Synergistic interaction between topotecan and microtubule-interfering agents. Cancer Chemother Pharmacol 48(3) (2001) 188-196).

Evidence of significant variation of CI as a function of drug ratio was observed particularly at low drug concentrations (low fₐ) for Daoy cells (FIG. 1B) and at high drug concentration for SW480 cells (FIG. 1D). Strong antagonism, reflected by high CI, values was seen for NB-EB cells (FIG. 1C).

In addition, strong antagonism reflected by CI values >3 was observed in Daoy cells at TPT/VCR mole ratio of 73, whereas synergy was observed at mole ratio of 2.9. A similar trend of ratio-dependent synergy was observed for SW480 cells (FIG. 1D), where antagonism was evident at TPT/VCR ratios of 0.2, 3.7, and 18.3, and strong synergism (CI<0.5) was evident at a TPT/VCR ratio of 0.7. The highest degree of drug ratio dependency was observed in SW480 colon cancer tumor line.

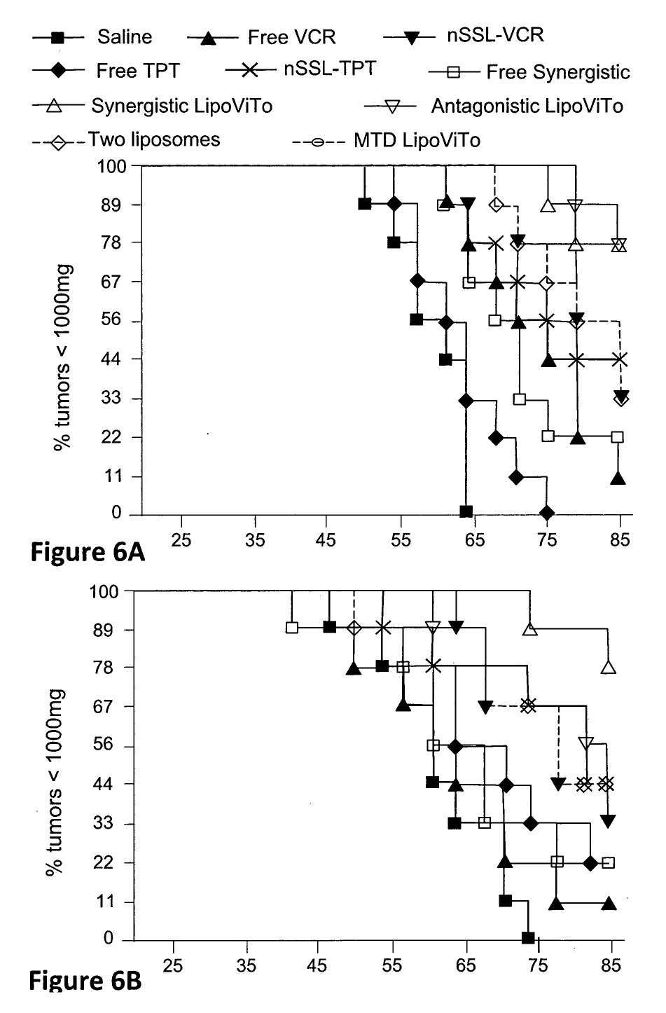

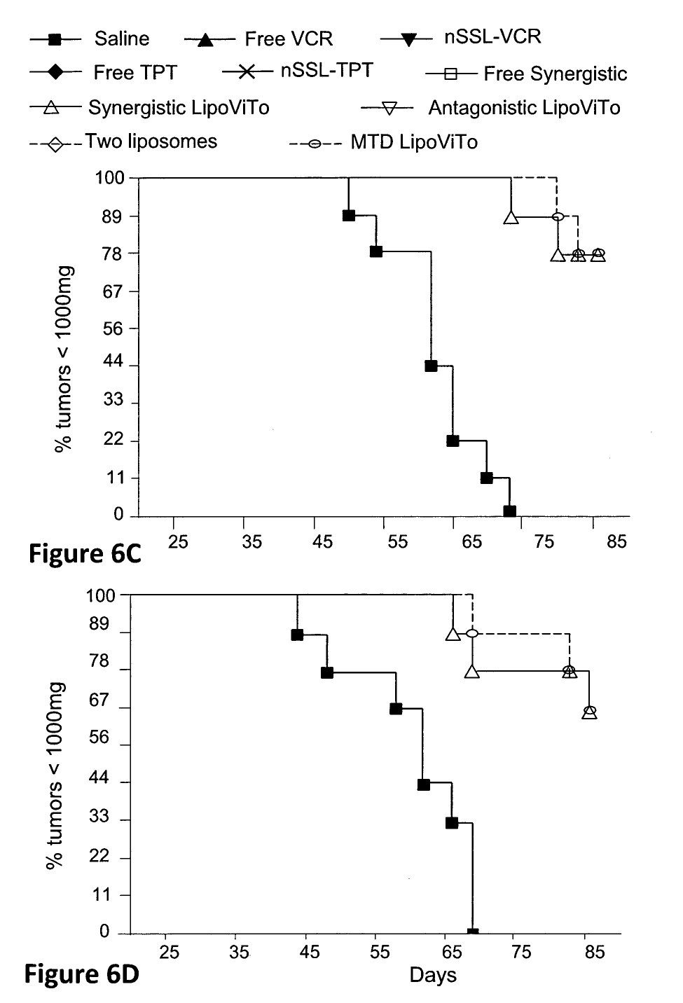

The liposomes co-encapsulating the drugs with MTD drug ratio were more or equally efficacious as compared to the same liposomes with antagonists or synergistic drug ratio (FIGS. 6A-6D).

### VCR and TPT Co-Encapsulation in Liposomes

The need to develop one liposome that includes two drugs at a specific predefined mole ratio required optimization of the loading conditions and “crosstalk” with the drugs. For this, physiochemical characterization of the two drugs is required. The amphipathic nature of the two weak basses VCR and TPT is strongly pH dependent as shown in Table 3.

TABLE 3
Physicochemical properties of the two weak basses VCR and TPT

| | | | | | Non- |
|---|---|---|---|---|---|
| | | | | | polar/ |
| | | | | | polar |
| | | | | | sur- |
| | | | | | face |
| | | Solubility | | | area |
| Drug | pKₐ | (mM) [pH] | logP | logD [pH] | ratio |
| VCR | 7.64, 6.81 | 0.3 [6], 0.01 [8] | 2.97 | 1.2 [6], 2.81 [8] | 3.62 |
| TPT | 7.65 | 3.6 [6], 0.2 [8] | 1.39 | −0.27 [6], 1.00 [8] | 2.81 |

When the pH decreases below the pKₐ of these drugs, their amino group becomes protonated. This protonation leads to an increase in the drug's solubility and decrease in its logD.

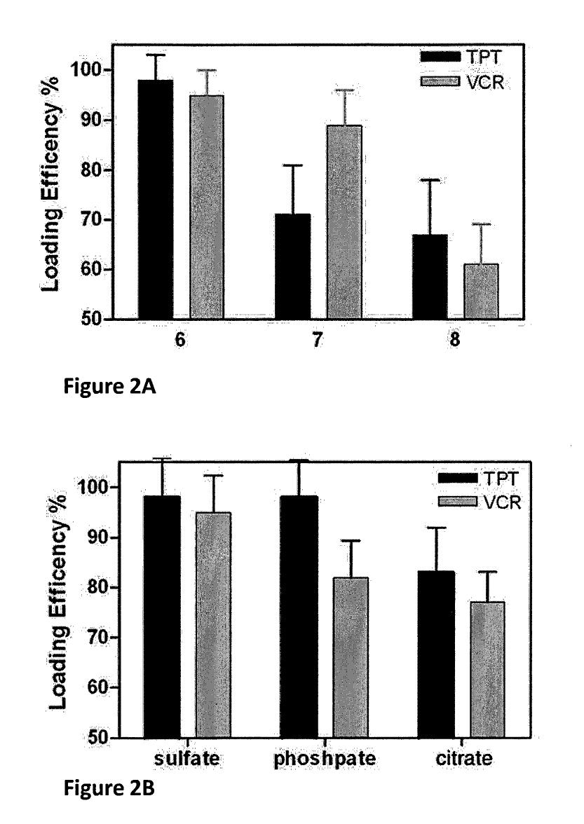

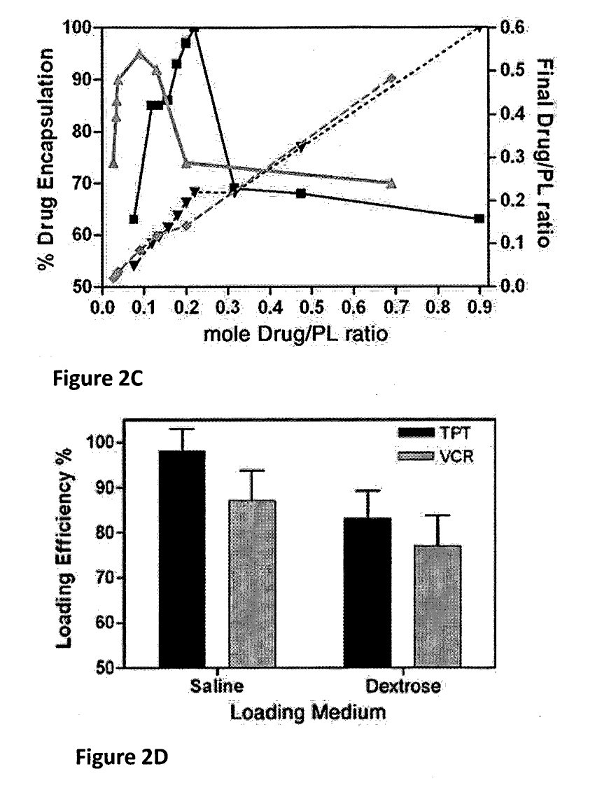

In remote loading, the drug to be encapsulated is introduced to the aqueous medium containing preformed nSSLs [Y. Barenholz, Relevancy of drug loading to liposomal formulation therapeutic efficacy. J. Liposome Res. 13(1) (2003) 1-8]. The effect of external medium pH was studied by encapsulating drugs at different medium pHs (FIG. 2A), concluding that the optimal loading for both drugs is achieved at pH 6.

Based on the inventors' previous experience with remote loading of amphipathic weak bases, such as doxorubicin [Y. Barenholz, Relevancy of drug loading to liposomal formulation therapeutic efficacy. J. Liposome Res. 13(1) (2003) 1-8; G. Haran, R. Cohen, L. K. Bar, Y. Barenholz, Transmembrane ammonium sulfate gradients in liposomes produce efficient and stable entrapment of amphipathic weak bases. Biochim. Biophys. Acta 1151(2) (1993) 201-215], in order to achieve a stable enough loading, the ion which is directly responsible for the loading (NH₄⁺) needs “help” from its counteranion. The mechanism of stabilization is associated with intra-liposome drug-counteranion salt precipitation. Thus, the effect of the counteranion on the loading was also characterized. (FIG. 2B). It was found that, in terms of the highest encapsulation efficiency, the optimal counter ion for both these drugs was sulfate. It is essential for simultaneous loading that the drugs are compatible with the same countarion, i.e. that both are stabilized by the same counteranion.

Without being limited thereto, the superiority of sulfate as a counteranion can be explained by its low membrane permeability and its low solubility product, which stabilizes drug accumulation of the drug-sulfate salt [V. Wasserman, P. Kizelsztein, O. Garbuzenko, R. Kohen, H. Ovadia, R. Tabakman, Y. Barenholz, The antioxidant tempamine: in vitro antitumor and neuroprotective effects and optimization of liposomal encapsulation and release. Langmuir 23(4) (2007) 1937-1947]. These salts also differ in the ionic strength of their anion, (having the following order: (HSO₄⁻), SO₄⁻²≈HPO₄⁻², (PO₄⁻³)>citrate⁻³), as well as in the charge of the anion.

The optimal drug-to-phospholipid (PL) mole ratio at the beginning of loading was evaluated by measuring the drug encapsulation at different drug/PL ratios used for the loading (FIG. 2C). The results show that, in terms of the highest encapsulation efficiency, the optimal drug-to-PL mole ratio was ˜0.220 and ˜0.1 for TPT and VCR, respectively. Above these ratios, there was a decline in encapsulation efficiency. Since VCR is much more potent than TPT, its required drug-to-PL ratio would be much lower.

Further, it was found that saline, as an extra liposome medium, enabled achieving a better loading than dextrose 5% (FIG. 2D)

These data presented in FIGS. 2A-2D served as the basis for the loading of both drugs simultaneously at the desired ratios: synergistic and antagonistic (Table 4).

### Loading Conditions:

External medium: saline at pH 5.7;

Gradient forming salt: ammonium sulfate,

Loading duration: 30 min at 55° C.

The same preliminary analysis can be conducted for any combination of weak amphipathic drugs for which co encapsulation is desired, so as to determined the optimal formulation of the selected two or more drugs.

The clinical doses of both drugs are low, remote co-loading of them at the desired drug-to-PL ratios was thus achieved without lowering the loading efficacy and nSSL capacity. It was further found that the loading of one drug at the determined optimal conditions did not interfere with the loading of the other drug. Such interference occurred when a higher drug/PL ratio was used. For instance, at VCR/PL mole ratio of 0.69, VCR loading decreased from −70% to −30% at 0.69 VCR/PL mole ratio due to addition of 0.43 TPT/PL mole ratio.

TABLE 4
Loading of TPT and VCR at synergistic and antagonistic ratios

| | Mole | | | | |
|---|---|---|---|---|---|
| | drug-to-PL | | Drug | | Mole |
| | ratio | | loading % | | TPT/VCR |
| Formulation | TPT | VCR | TPT | VCR | ratio |
| Liposomal TPT | 0.20 | | 98 | | |
| Liposomal VCR | | 0.130 | | 92 | |
| Daoy synergistic-LipoViTo | 0.20 | 0.068 | 98 | 95 | 2.9 |
| Daoy antagonistic-LipoViTo | 0.20 | 0.003 | 98 | 95 | 73 |
| SW480 synergistic-LipoViTo | 0.19 | 0.031 | 98 | 95 | 0.7 |
| SW480 antagonistic-LipoViTo | 0.02 | 0.027 | 100 | 95 | 18.3 |

Cryo-TEM of nSSL-VCR, nSSL-TPT, and nSSL co-loaded with VCR and TPT (LipoViTo) confirmed the size distribution as determined by dynamic light-scattering (DLS). It showed that all three nSSL formulations had spherical shapes as for unloaded nSSL. However, the drug loaded nSSL differed in their content: The interior of nSSL-VCR (FIG. 3B) appeared more electron-dense than the drug free control nSSL (FIG. 3A). This suggested an amorphous VCR-sulfate precipitation. This morphology was different from liposomal TPT (FIG. 3C), whose drug nano-crystals were clearly seen in the liposome interior. LipViTo (FIG. 3D) looked like an overlay of nSSL-VCR and nSSL-TPT (of FIGS. 3B and 3C).

nSSL-Drug Stability And Drug Release

Physical stability of nSSL-drug is highly important for product shelf life. Therefore, the physical stabilities of nSSL-TPT, nSSL-VCR and LipoViTo were followed at 4° C. for six months. In all nSSL drugs release during six months was below detection limits. The size distribution of the liposomes did not change during storage at 4° C. as examined by dynamic light scattering (DLS). Further, after six months storage at 4° C. the liposomal formulations were analyzed by and TLC. For HPLC, VCR was detected using UV detector at 220 nm, TPT with fluorescence detector and an excitation/emission wavelengths of 416/522 and HSPC with cholesterol with ELSD detector at 50° C. and 1.3 L/min gas flow and a UV detector at 254 nm. For TLC a mobile phase of chloroform:methanol:water (85:15:1.5 v/v/v) was used on a silica plate. The HPLC and TLC analyses showed that the liposomal drug formulations contained only intact drugs, HSPC and cholesterol (data not shown).

Without being bound by theory, this may be attributed, inter alia, to the selection of a rigid liposome forming lipid, HSPC which lead to a lipid bilayer at rigid liquid ordered phase, and its combination with cholesterol, DSPE-2 kPEG and remote loading. This supports low release energy at storage under 4° C. but sufficient to achieve therapeutic release and activity at 37° C., as discussed below.

The release of the drugs in vitro at 37° C. was studied by incubating nSSL-TPT, nSSL-VCR and LipoViTo in serum at 37° C., which was relevant to in vivo situation. Drug release was slow for both drugs, and most of the drug was released after 4 days. The release rate is similar for nSSL-TPT, nSSL-VCR and LipoViTo (encapsulating both drugs) (FIG. 4).

VCR release was linear, characterized by zero-order kinetics, while TPT release was characterized by a combination of first-order kinetics followed by zero-order kinetics. VCR release rate (t_{1/2}≈81 h) was slower than TPT release rate (t_{1/2}≈55 h), and both had a similar pharmacokinetics to Doxil™[A. Gabizon, H. Shmeeda, Y. Barenholz, Pharmacokinetics of Pegylated Liposomal Doxorubicin: Review of Animal and Human Studies. Clinical Pharmacokinetics 42 (2003) 419-436]. The release rates of nSSL-VCR were slower than nSSL-VCR loaded by MgSO₄ gradient (t_{1/2}≈4 h) [I. V. Zhigaltsev, N. Maurer, Q. F. Akhong, R. Leone, E. Leng, J. Wang, S. C. Semple, P. R. Cullis, Liposome-encapsulated vincristine, vinblastine and vinorelbine: a comparative study of drug loading and retention. J. Control. Release 104(1) (2005) 103-111].

Pharmacokinetic study with PEGylated liposomal VCR and TPT in mice in which the fates of ¹⁴C CHE liposome label and the drugs are measured in plasma. The drug release was calculated from the decrease in the drug-to-liposome ratio.

The release rate of a drug from liposome with a single agent is very similar to the release rate of the same drug from LipoViTo.

### Pharmacokinetics

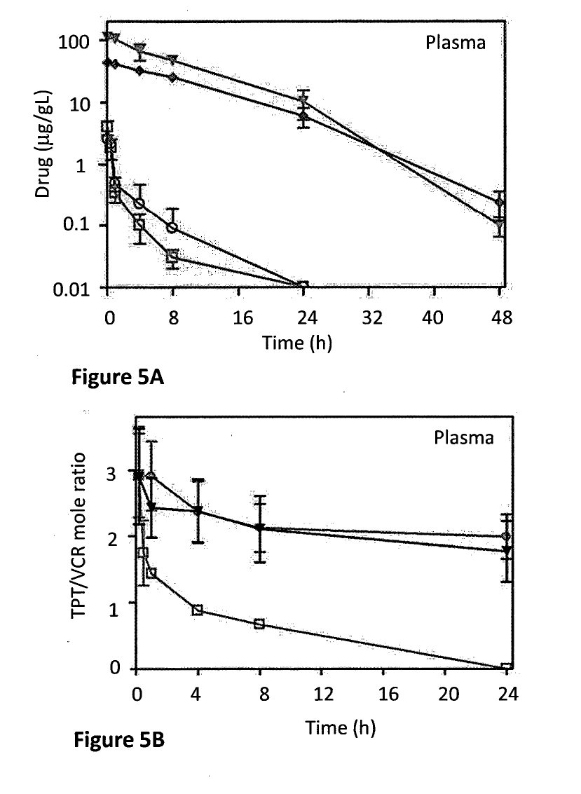

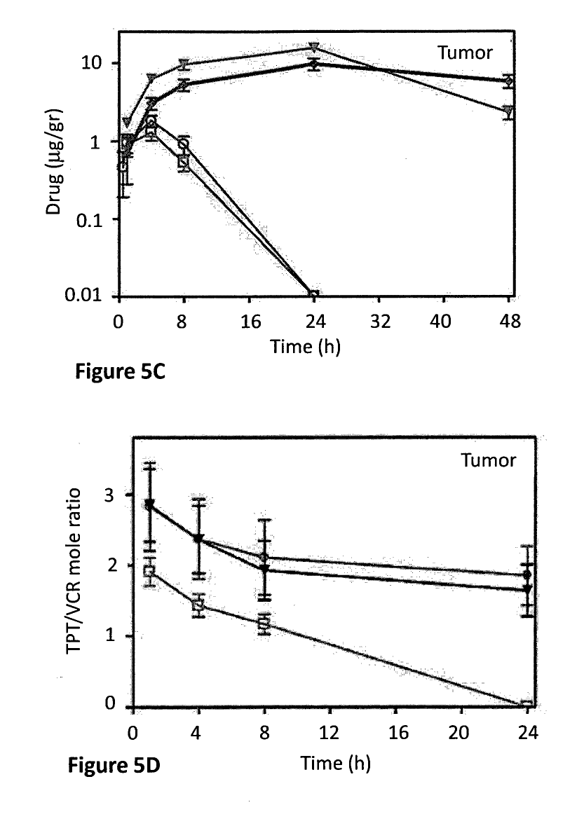

The pharmacokinetics of VCR and TPT after the administration of free drugs or liposomal drugs is shown in FIG. 5A. Key pharmacokinetic parameters were calculated from these data and are presented in Table 5.

TABLE 5
Tumor-bearing nude mice serum pharmacokinetic parameters comparing free
drugs and liposomal drugs.

| | | Formulation | | | |
|---|---|---|---|---|---|
| | | | | Liposomal | Liposomal |
| Parameter | Units | Free TPT | Free VCR | TPT | VCR |
| Dose | mg/kg | 10 | 2 | 5 | 2 |
| AUC¹ | h × μg/ml | 2.7 ± 0.91 | 2.51 ± 0.46 | 1232 ± 141.5 | 769.2 ± 90.6 |
| t_{1/2}² | h | 1.03 ± 0.14 | 2.09 ± 0.29 | 7.05 ± 1.37 | 6.94 ± 0.83 |
| Cₘₐₓ³ | μg/ml | 3.98 ± 1.04 | 1.37 ± 0.34 | 123.75 ± 14.17 | 49.5 ± 5.14 |
| CL⁴ | ml/h | 76.93 ± 8.21 | 16.36 ± 2.61 | 0.08 ± 0.02 | 0.06 ± 0.01 |
| MRT⁵ | h | 0.6 ± 0.07 | 1.73 ± 0.3 | 6.63 ± 0.71 | 12.52 ± 1.33 |
| Vss⁶ | ml | 65.92 ± 7.21 | 38.24 ± 9.68 | 0.74 ± 0.17 | 0.72 ± 0.11 |

¹Area under the concentration time curve.
²The elimination half life, which is the time taken for plasma concentration to be reduced by 50%.
³The maximum observed concentration in the plasma.
⁴Clearance.
⁵Mean residence time.
⁶Volume of drug distribution at steady state.

Free drugs were rapidly eliminated from the plasma. Their area under the time curve (AUC), half-life, mean resistance time (MRT), and Cmax values were significantly lower, whereas volume at steady state (Vss) values were significantly higher, than those of liposomal formulations. For instance, the half life values were 1.03, 2.09, 7.05 and 6.94 h for free TPT, free VCR, liposomal TPT and liposomal VCR, respectively. After the administration of either liposomal formulation, elevated plasma and tumor concentrations of VCR and TPT were maintained up to 48 h post injection (FIGS. 5A and 5C). Two days post liposomal administration; there were significant levels of both drugs in the tumor, whereas plasma levels were very low. Thus, both drugs were delivered efficiently to the tumors by the liposomes.

The higher Cmax values, longer circulation half-lives, and longer mean residence times observed with the liposomal formulations, compared with free drugs, were associated with significantly higher plasma AUC values. The AUC values for liposomal VCR (769 μg×h/ml) and liposomal TPT (1232 μg×h/ml) were 306- and 456-fold greater than that for the free VCR and free TPT. Taken together, these data are similar to those described previously for both liposomal TPT and liposomal VCR [Y. Hao, Y. Deng, Y. Chen, K. Wang, A. Hao, Y. Zhang, In-vitro cytotoxicity, in-vivo biodistribution and antitumour effect of PEGylated liposomal topotecan. J. Pharmacy and Pharmacol. 57(10) (2005) 1279-1288; R. Krishna, M. S. Webb, G. St Onge, L. D. Mayer, Liposomal and nonliposomal drug pharmacokinetics after administration of liposome-encapsulated vincristine and their contribution to drug tissue distribution properties. J Pharmacol Exp Ther 298(3) (2001) 1206-1212]. Although free VCR's t_{1/2} is ˜2 times greater that TPT's t_{1/2}, the t_{1/2}, of liposomal VCR and liposomal TPT is very similar (Table 5).

The lactone-protecting effect in-vivo was also observed. Eight hours post injection, the lactone ratio of TPT for liposomal TPT increased to 76%, compared with the lactone ratio of 9% for free TPT based on AUC value. Without being bound by theory, this may be due to two independent effects:
The significant in vivo protection of the lactone ring of encapsulated TPT from hydrolysis by the liposomes.
The acidic intraliposomal environment, which resulted from the transmembrane ammonium sulfate gradient.

HPLC and TLC analyses showed that the circulating liposomal drug formulations contained intact drug; no evidence of degradation was observed for both drugs (discussed above).

Daoy synergistic-LipoViTo formulation maintained the TPT/VCR mole ratio in the range of 2.9-2 over extended times (up to 24 hours) in plasma and tumor after i.v. injection into mice (FIGS. 6B and 5D). However, upon injection of free drugs at the same ratio, the ratio declined rapidly (in 2 hours from 2.9 to <1.0) due to the higher clearance of TPT.

Therapeutic Efficacy of VCR, TPT and their Liposomal Formulations in Solid Tumor Models

Kaplan-Meier curves (E. L. Kaplan, P. Meier, Nonparametric estimates from incomplete observations. J. Am. Stat. Assoc 53(282) (1958) 457-481] were employed in order to describes results of the in vivo efficacy studies. The mice were scarified when their tumors reach a size of ≧1000 mg. Therefore, this event was chosen as the endpoint of the construction of the curves. It is an analogue to survival.

Treatment of established Daoy (medulloblastoma) tumors with the formulations of LipoViTo yielded therapeutic activity with tumor regression at synergistic and antagonistic drugs ratios (FIG. 6A).

The activity was significantly greater than treatment by nSSLs with one agent, singly or in combination as shown in Table 6.

TABLE 6
Statistical Significance between the different treatments in FIG. 6
determined using Logrank test

| | | One tail p- | Two tails |
|---|---|---|---|
| FIG. | Compared treatments | value | p-value |
| 6A | Synergistic LipoViTo ≠ Anatgonistic LipoViTo | | 0.9253 |
| 6A | synergistic LipoViTo > nSSL TPT | 0.0563 | |
| 6A | synergistic LipoViTo > nSSL VCR | 0.0405 | |
| 6A | synergistic LipoViTo > two liposomes | 0.0352 | |
| 6A | nSSL-VCR ≠ nSSL-TPT ≠ two liposomes | | 0.9879 |
| 6A | nSSL-VCR > free VCR | 0.0642 | |
| 6A | nSSL-TPT > free TPT | 0.0014 | |
| 6A | free VCR > saline | 0.0005 | |
| 6A | free TPT > saline | 0.0904 | |
| 6A | free synergistic drugs > saline | 0.0011 | |
| 6A | free VCR > free TPT | 0.0054 | |
| 6B | synergistic LipoViTo > nSSL TPT | 0.0622 | |
| 6B | synergistic LipoViTo > Antagonistic LipoViTo | 0.0626 | |
| 6B | synergistic LipoViTo > two liposomes | 0.0622 | |
| 6B | synergistic LipoViTo > nSSL VCR | 0.0252 | |
| 6B | Antagonistic LipoViTo ≠ nSSL-TPT ≠ two liposomes | | 0.9971 |
| 6B | nSSL-VCR > free VCR | 0.0337 | |
| 6B | nSSL-TPT > free TPT | 0.0706 | |
| 6B | free VCR > saline | 0.2176 | |
| 6B | free TPT > saline | 0.0357 | |
| 6B | free synergistic drugs > saline | 0.1211 | |
| 6B | free TPT > free TPT | 0.1850 | |
| 6C | Synergistic LipoViTo ≠ MTD LipoViTo | | 0.9253 |
| 6C | Synergistic LipoViTo > saline | <0.0001 | |
| 6C | MTD LipoViTo > saline | <0.0001 | |
| 6D | MTD LipoViTo > Synergistic LipoViTo | 0.4693 | |
| 6D | Synergistic LipoViTo > saline | <0.0001 | |
| 6D | MTD LipoViTo > saline | <0.0001 | |

As shown in FIG. 6A, the nSSLs with single drug were more efficacious than treatment with free drugs. Treatment with the free drugs (VCR or both drugs at synergistic ratio) was better than treatment with saline.

Treatment of established SW480 (colon) tumors was most efficacious by synergistic-LipoViTo (FIG. 6B). A mixture of nSSL-TPT and nSSL-VCR at synergistic ratio, nSSL-TPT, and antagonistic-LipoViTo had similar therapeutic efficacies, which were inferior to synergistic-LipoViTo. Free TPT and free drugs at synergistic ratio had similar therapeutic efficacy, which were inferior to the liposomal formulations. Treatment with free was better than treatment with saline.

Free VCR was more efficacious for treatment of medulloblastome than free TPT, while free TPT was more efficacious for colon cancer (FIGS. 6A and 6B).

Next, LipoViTo with both drugs at the ratio corresponding to their MTDs (TPT mg/kg and VCR 1.5 mg/kg, TPT/VCR mole ratio of 7.3) were prepared in order to compare their therapeutic efficacy with the appropriate synergistic-LipoViTo. VCR dosage was reduced from 2 mg/kg to 1.5 mg/kg in order to avoid toxicity problems due to combination with the high dosage of TPT. Treatment of Daoy and SW480 cancers with MTD-LipoViTo and synergistic-LipoViTo resulted in similar efficacies (FIGS. 6C and 6D).

Treatment of SW480 cancer was slightly more efficacious with MTD-LipoViTo than with synergistic-LipoViTo (FIG. 6D), although this difference was not statistically significant.

1-47. (canceled)

48. A liposome having co-encapsulated in its intraliposomal core at least two amphipathic drugs, the at least two amphipathic drugs being either at least two amphipathic weak base drugs or at least two amphipathic weak acid drugs, the at least two amphipthic drugs being within the intraliposomal core, wherein,
the at least two amphipathic drugs are co-encapsulated in the liposome at a pre-determined maximal tolerated dose (MTD) ratio;
the liposome comprises one or a combination of liposome forming lipids, the one or combination of liposome forming lipids have a solid ordered (SO) to liquid disordered (LD) phase transition temperature above 37° C.;
each of the at least two amphipathic drugs exhibit, when co-encapsulated in the same liposome, a liposomal profile that corresponds to the profile of each drug when encapsulated as a single drug in the same liposome;
wherein the at least two amphipathic drugs are selected to exhibit different mechanism of actions, and
the liposome is absent of one or both of a transition metal and a ionophore.

49. The liposome of claim 48, wherein each of the amphipathic drug is an anti cancer drug exhibiting a different mechanism of action against the cancer.

50. The liposome of claim 48, comprising a concentration of each drug in the intraliposomal aqueous core that is either greater than the maximal solubility of the drug in water or is above 50 nM.

51. The liposome of claim 48, comprising a counter ion compatible to the at least two amphipathic drugs.

52. The liposome of claim 48, wherein the at least two amphipathic drugs are present in the intraliposomal aqueous core of the liposome in free form or in precipitated salt form with the counter ion.

53. The liposome of claim 48, comprising at least one phospholipid in combination with a lipopolymer.

54. The liposome of claim 48, comprising cholesterol.

55. The liposome of claim 48, having a size of between 20 nm to 150 nm.

56. The liposome of claim 48, exhibiting in vivo a, time dependent, controlled release profile for each of said amphipathic drug.

57. The liposome of claim 49, wherein each of the amphipathic drug exhibit a different mechanism of action, the mechanism of action being selected from the group consisting of antimetabolites, DNA damaging agent, topoisomerase I inhibitors, topoisomerase II inhibitors, alkylating agents, DNA synthesis inhibitors, apoptosis inducing agent, cell cycle inhibitor, anti-mitotic agents, anti-angiogenesis agent and anticancer antibiotics.

58. The liposome of claim 48, wherein the at least two amphipathic drugs are selected from anthracyclines, camptothecins, glucocorticoids, plant alkaloids, and vincalkaloids.

59. The liposome of claim 48, wherein one of the at least two amphipathic drugs is a camptothecin and the other of the at least two amphipathic drugs is a vincalkaloid.

60. The liposome of claims 48, comprising two amphipathic drugs, a first amphipathic drug being topotecan and a second amphipathic drug being vincristine.

61. A pharmaceutical composition comprising a physiologically acceptable carrier and liposomes according to claim 48.

62. A pharmaceutical composition comprising a physiologically acceptable carrier and liposomes according to claim 49.

63. A method for simultaneous co-enacpsulation into a liposome of at least two amphipathic drugs, the method comprising:
(a) providing a suspension of liposomes comprising in the intraliposomal aqueous core of the liposome a weak acid or weak base and a counter ion of the weak acid or weak base, the concentration of the weak acid or weak base being greater inside the liposome than outside the liposome;
(b) simultaneously incubating the liposomes with at least two amphipathic drugs having a pre-determined MTD ratio therebetween, the at least two amphipathic drugs being compatible with the counter ion, wherein, when the liposomes comprise a weak acid, the at least two amphipathic drugs are weak amphipathic acid drugs, and when the liposomes comprise a weak base, the at least two amphipathic drugs are weak amphipathic base drugs, wherein the at least two amphipathic drugs are selected to exhibit different mechanism of actions;
wherein,
the liposome comprises one or combination of liposome forming lipids, the one or combination of liposome forming lipids have a solid ordered (SO) to liquid disordered (LD) phase transition temperature above 37° C.;
the incubation is under conditions sufficient to allow simultaneous co-encapsulation in the intraliposomal aqueous core of the liposome of the two amphipathic drugs without use of a transition metal and the encapsulation is at a pre-determined MTD ratio between the at least two amphipathic drugs;
when in the liposome, each of the at least two amphipathic drugs exhibit a liposomal profile that corresponds to the profile of each drug when encapsulated as a single drug in the same liposome; and
for each drug, the method provides a loading efficiency above 85%.

64. The method of claim 63, wherein the suspension of liposomes comprise pre-formed liposomes having a lower inside/higher outside H⁺ or ion gradient.

65. The method of claim 63, wherein the conditions sufficient to allow simultaneous co-encapsulation of the two amphipathic drugs in the pre-determine ratio are selected from external pH, type of loading medium and type of counter ion.

66. The method of claim 63, comprising incubation under conditions sufficient to allow at least part of the amphipathic drug to precipitate within the intraliposomal core.

67. The method of claim 63, wherein the at least two amphipathic drugs are anti-cancer drugs selected to exhibit different mechanism of actions against cancer.

68. The method of claim 63, wherein the concentration of each drug in incubation is greater than the maximal solubility of each drug in water, thereby providing a loading efficiency into said liposomes of at least 90%.

69. A method of treatment of a subject comprising administering to the subject a pharmaceutical composition comprising liposomes according to claim 48.

70. The method of claim 69 comprising systemic administration of the pharmaceutical composition to the subject in need thereof.

71. The method of claim 69, wherein the at least two amphipathic drugs are anti-cancer drugs selected to exhibit different mechanism of actions against cancer.
